(19) United States
(12) Patent Application Publication
(10) Pub. No.: US 2007/0166367 A1
(43) Pub. Date: Jul. 19, 2007

(54) Adept or gdept producing acetaldehyde

(76) Inventor: Philip Savage, London (GB)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/588,626

(22) PCT Filed: Feb. 3, 2005
(86) PCT No.: PCT/GB05/00363

§ 371 Date: Aug. 4, 2006

(30) Foreign Application Priority Data
Feb. 6, 2004 (GB) 0402626.6
Jun. 22, 2004 (GB) 0413975.4

(51) Int. Cl. A61K 39/395; A61K 9/127

(52) U.S. Cl. 424/450; 424/146.1; 424/155.1

(57) ABSTRACT

The present invention includes compounds, comprising a portion capable of converting a substrate, for example ethanol, to acetaldehyde and a means of directing the compounds to selected cells, and their use in therapeutic compositions and methods. The compounds may be administered with the substrate, for example ethanol, and an inhibitor of aldehyde dehydrogenase.

This application is a U.S. National Phase Application pursuant to 35 U.S.C. 371 of International Application No. PCT/GB2005/000363, which was filed Feb. 3, 2005, claiming benefit of priority of Great Britain Patent Application No. 0402626.6, which was filed Feb. 6, 2004, and Great Britain Patent Application No. 0413975.4, which was filed Jun. 22, 2004. The entire disclosure of each of the foregoing applications is incorporated herein by reference.

This invention relates to therapeutic systems, particularly therapeutic systems for targeting cytotoxic treatment to cells, particularly tumour cells.

The delivery of cytotoxic treatment to the site of tumour cells is much desired, because systemic cytotoxic treatment can result in the killing of normal cells within the body as well as tumour cells. This limits the intensity and duration of cytotoxic treatment that can be administered and thus reduces the therapeutic potential of the treatment.

In some known therapeutic systems, including known cytotoxic treatment systems, the administered agent has no intrinsic activity but is converted in vivo at the appropriate time or place to the active agent. Such agents are known as pro-drugs, and are used extensively in medicine (Connors and Knox, (1995), Expert Opinion on Therapeutic Patents 5, 873-885). Conversion of the pro-drug to the active form can take place by a number of mechanisms depending, for example, on changes of pH, oxygen tension, temperature or salt concentration or by spontaneous decomposition of the pro-drug or internal ring opening or cyclisation.

W088/07378 describes a two-component system, and therapeutic uses thereof, wherein a first component comprises an antibody fragment capable of binding with a tumour-associated antigen and an enzyme capable of converting a pro-drug into a cytotoxic drug, and a second component which is a pro-drug which is capable of conversion to a cytotoxic drug. This general system, which is often referred to as “antibody-directed enzyme pro-drug therapy” (ADEPT), is also described in relation to specific enzymes and pro-drugs in EP 0 302 473 and WO 91/11201.

W089/10140 describes a modification to the system described in W088/07378 wherein a further component is employed in the system.

This further component accelerates the clearance of the first component from the blood when the first and second components are administered clinically. The second component is usually an antibody that binds to the antibody-enzyme conjugate and accelerates clearance. An antibody which was directed at the active site on the enzyme had the additional advantage of inactivating the enzyme. However, such an inactivating antibody has the undesirable potential to inactivate enzyme at the tumour sites, but its penetration into tumours was obviated by the addition of galactose residues to the antibody. The galactosylated antibody was rapidly removed from the blood, together with bound antibody-enzyme component, via galactose receptors in the liver. The system has been used safely and effectively in clinical trials. However, galactosylation of such an inactivation antibody which results in its rapid clearance from blood also inhibits its penetration of normal tissue and inactivation of enzyme localised there.

WO 93/13805 describes a system comprising a compound comprising a target cell-specific portion, ie. a portion which specifically binds target cells such as an antibody specific to tumour cell antigens, and an inactivating portion, such as an enzyme, capable of converting a substance which in its native state is able to inhibit the effect of a cytotoxic agent into a substance which has less effect against said cytotoxic agent. The prolonged action of a cytotoxic agent at tumour sites is therefore possible whilst protecting normal tissues from the effects of the cytotoxic agent.

W093/13806 describes a further modification of the ADEPT system comprising a three component kit of parts for use in a method of destroying target cells in a host. The first component comprises a target cell-specific portion and an enzymatically active portion capable of converting a pro-drug into a cytotoxic drug; the second component is a pro-drug convertible by said enzymatically active portion to the cytotoxic drug; and the third component comprises a portion capable of at least partly restraining the component from leaving the vascular compartment of a host when said component is administered to the vascular compartment, and an inactivating portion capable of converting the cytotoxic drug into a less toxic substance.

EP 0 415 731 describes a therapeutic system which is often called GDEPT 15 (gene-directed enzyme pro-drug therapy). In this system, a composition comprising a polynucleotide encoding the activating enzyme is administered instead of the activating enzyme itself.

WO 87/03205 describes a therapeutic system in which the substrate of an antibody-targeted enzyme is a substance close to, or part of, the targeted cell or antigen, or the fluid surrounding the cell or antigen. Examples include glucose oxidase, which acts on glucose present in plasma; one of the products is hydrogen peroxide which can kill cells by oxidation of components of the cell wall.

Glucose oxidase targeting was also described by Philpott et al ((1973) J. Immunol. 111, 921-929) and reviewed in Senter et al (1993) Bioconjugate Chem. 4, 3-9. Limited cytotoxicity was observed with glucose oxidase conjugates, but when the glucose oxidase reaction was coupled to lactoperoxidase and iodide, iodine was formed and significant cytotoxicity was observed. This system is developed further in Parker et al (1975) PNAS USA 72(1), 33 8-342, wherein luminol-glutathione conjugates of glucose oxidase are used in a two-step targeting system: an antibody horseradish peroxidase conjugate is targeted to cells, where it oxidises the luminol to form a free radical that reacts with cell components to anchor the glucose oxidase, which then generates the cytotoxic hydrogen peroxide as previously described.

New systems of treatment are needed. Alternative systems of treatment are needed. Problems with the prior art include side effects.

The concept of targeted enzyme pro-drug systems that convert a relatively non-toxic agent into a potent cytotoxic drug at the site of the tumor or other target cells has been described (Philpott et al 1973). Theoretically this approach should offer an elegant method to selectively deliver cytotoxic therapies either as a gene mediated (GDEPT) or antibody (ADEPT) based systems, but has been limited by a number of issues including, prodrug stability and toxicity, lack of localization, poor levels of enzyme activity and the limited toxicity of active drugs particularly those which are cell cycle specific.

Prodrugs that are broken down by cellular (untargeted) enzymes to release formaldehyde are known, for example triazenes, natulan and hexamethylmelamine (reviewed in Connors (1976) in Progress in Drug Metabolism, Bridges & Chasocaud, Eds.), but it is not clear whether formaldehyde release is the method of action.

Philpott et al (1979) Cancer Res 39, 2084-2089 describes an alcohol dehydrogenase-antibody conjugate and its use in vitro with the substrate allyl alcohol to promote cytotoxicity. Several potential problems in applying the system in vivo are mentioned, particularly hepatotoxicity due to conversion of allyl alcohol to the toxic acrolein by endogenous liver alcohol dehydrogenase.

### SUMMARY OF THE INVENTION

An object of this invention is to exploit the local toxicity of acetaldehyde, and/or to provide means to produce it locally within tumours as a therapeutic strategy that and/or cause direct cytotoxicity to occur in target cells via acetaldehyde and/or to produce an increase in the immunogenicity of target cells such as tumour cells.

The invention provides a therapeutic system which delivers cytotoxic therapy to the site of target cells by generation of acetaldehyde in the fluid surrounding the target cells. This system uses a pro-drug (for example, ethanol or pyruvate) that requires no special synthesis and is essentially non-toxic even in very high concentration (judged against conventional pharmacology agents). Additionally the active toxin may be a normal metabolite of the body (i.e. acetaldehyde produced from ethanol), not a synthetic drug, for example a chemotherapy drug. The acetaldehyde may exert a toxic effect as a consequence of its concentration and location of production. It will be appreciated that unwanted effects (side effects) of a toxin that is a normal metabolite of the body are advantageously less than the unwanted effects of a molecule that is not a normal metabolite.

The present invention includes compounds or systems, comprising an acetaldehyde forming portion and a means of directing the compounds to selected cells, and their use in therapeutic compositions and methods. The compounds or systems may be administered with a substrate that is converted to acetaldehyde by the acetaldehyde forming portion, and optionally with a substantially reversible inhibitor of the acetaldehyde forming portion, such that activity of the acetaldehyde forming portion is inhibited until the compounds are substantially localised at the selected cells. Optionally, an inhibitor of aldehyde dehydrogenase may also be administered. Thus the invention includes a therapeutic system in which a targeted enzyme, for example alcohol dehydrogenase, acts on a substantially non-toxic substrate, for example ethanol, to produce local cytotoxic conditions.

As an example, acetaldehyde may be produced locally in tumours as a result of the presence of the enzyme alcohol dehydrogenase within the tumour targeted there by either antibody delivery systems, liposomes or gene therapy delivery systems, or any other suitable system to allow the localisation of a suitable enzymatically active component at the tumour site.

Clearly, the term ‘compound’ as used herein embraces entities whose component parts are bonded together eg. covalently or ionically or other such bonding. Furthermore, the term ‘compound’ as used herein also embraces lower order associations such as hydrogen bonding or affinity interaction such as can occur between associating molecules such as avidin/biotin/streptavidin and the term ‘compound’ does not necessarily imply a covalent connection between each element of said compound. The application as a whole makes this clear.

A first aspect of the invention provides a method of damaging and preferably destroying target cells in a host/subject, the method comprising administering to the host/subject
(1) a compound comprising a target-cell specific portion and a portion capable of converting a substrate to acetaldehyde; or a compound comprising a polynucleotide comprising a target cell-specific promoter operably linked to a polynucleotide encoding a polypeptide capable of converting a substrate to acetaldehyde; or a system for targeting a portion capable of converting a substrate to acetaldehyde to a target cell comprising (i) a target-cell specific portion further comprising a lock component and (ii) an portion capable of converting a substrate to acetaldehyde further comprising a key component that interacts specifically and with high affinity with the lock component or with an adapter component that interacts specifically and with high affinity with both the lock and the key component; and
(2) a substrate which is converted to acetaldehyde by the portion capable of converting said substrate to acetaldehyde, and optionally (3) a component that is capable of inhibiting aldehyde dehydrogenase, wherein step (2) is optional when the portion capable of converting a substrate to acetaldehyde is an enzymatically active portion of pyruvate decarboxylase.

In this way, damaging of the target cells is advantageously achieved. Preferably said cells are destroyed or damaged so extensively as to be killed, rendered inviable or otherwise terminated. Preferably the cells are destroyed. In some aspects of the invention, the acetaldehyde treatment induces damage which is not of itself lethal to an intact target cell, but in combination with another damaging agent produces a killing effect. Therefore, in this scenario, the acetaldehyde production may indeed advantageously destroy or kill that target cell which had itself been modified or weakened by said other agent. Alternatively it may damage the cell to a degree whereby said other agent will catalyse the destruction/killing of the cell. This is discussed in more detail below.

The terms ‘host’ and ‘subject’ are used interchangeably. The terms should not be taken to imply a host in the sense of a host/parasite relationship, but merely to specify the organism to which the methods/compositions or other aspects of the invention are applied. In particular, use of the term ‘host’ still embraces aspects of the invention which do not involve viral or other organismal vector systems.

The adapter component may itself comprise several components.

Administration of the substrate may advantageously start only once the level of acetaldehyde producing activity in the extracellular fluid has declined to a desired value ie. a therapeutically acceptable level. By this is meant that the level of acetaldehyde producing activity that can be detected in a sample of extracellular fluid taken from a site not thought to contain targeted cells, is not equal to or more than a level thought to cause an elevation in acetaldehyde level sufficient to cause cell damage.

The portions of the compound may be linked in a covalent or a non-covalent manner. The portion capable of converting a substrate to acetaldehyde may be directly active, in that it comprises a molecule that acts on a substrate present in the cells or their microenvironment to cause an elevation of the acetaldehyde concentration of the microenvironment.

Preferably the substrate is exogenous (i.e. the substrate is a molecule which, although it may be present naturally in the body, may be administered to the patient to be treated). When the substrate is pyruvate (ie the portion capable of converting a substrate to acetaldehyde is a catalytically active portion of pyruvate decarboxylase), the substrate may preferably be endogenous, i.e. present naturally in the body, particularly in or in the vicinity of the target cells. For example, some cancer cells may have significantly elevated levels of pyruvate (see Newsholme & Board (1991) Adv Enzyme Regul 31, 225-246; Baggetto et al (1992) Biochemie 74, 959-974).

The said portion capable of converting a substrate to acetaldehyde may comprise a polypeptide. In an alternative embodiment, the portion capable of converting a substrate to acetaldehyde may be indirectly modulating, in that it comprises a polynucleotide encoding a polypeptide capable of converting a substrate to acetaldehyde.
Thus in another aspect the invention relates to a method of damaging target cells in a subject, the method comprising administering to the subject
   (1) a nucleic acid encoding a compound capable of converting a substrate to acetaldehyde; and
   (2) a substrate which is converted to acetaldehyde by the portion capable of converting said substrate to acetaldehyde, and optionally
   (3) a component that is capable of inhibiting aldehyde dehydrogenase,
wherein step (2) is optional when the portion capable of converting a substrate to acetaldehyde is an enzymatically active portion of pyruvate decarboxylase.

Preferably the nucleic acid is in the form of a viral vector, preferably a DNA based viral vector, preferably an adenovirus derived viral vector.

The portion may involve complementation such as the portion itself being only a part of the active complex, and/or catalysing the acetaldehyde production by co-operation or complementation. Preferably the portion is active per se.

The portion capable of converting a substrate to acetaldehyde may achieve elevated levels of acetaldehyde as a result of its enzymatic activity. Preferably, it achieves elevated levels of acetaldehyde as a result of its enzymatic activity. More preferably, the portion capable of converting a substrate to acetaldehyde is an enzymatically active portion with alcohol dehydrogenase activity. It is particularly preferred if the portion capable of converting a substrate to acetaldehyde is an enzymatically active portion of an alcohol dehydrogenase or catalase or a microsomal oxidase or pyruvate decarboxylase. Still more preferably, it is an enzymatically active portion of human alcohol dehydrogenase or human catalase or a human microsomal oxidase. Yet more preferably it is an enzymatically active portion of human alcohol dehydrogenase, most preferably of human alcohol dehydrogenase β2 (Bosron et al (1985) “Purification and characterization of human liver beta 1 beta 1, beta 2 beta 2 and beta Ind beta Ind alcohol dehydrogenase isoenzymes” Prog Clin Biol Res 174, 193-206; may also be termed the B₂B₂ homodimer). Alcohol dehydrogenase is suitable because it acts on a substrate (ethanol) that is easily synthesised and well tolerated at levels of 500-2500 mg/L to yield the toxic metabolite acetaldehyde. Catalase and microsomal oxidases also act on ethanol to yield acetaldehyde, but may also metabolise other alcohols to yield aldehydes.

Alternatively, the portion capable of converting a substrate to acetaldehyde may be an enzymatically active portion of pyruvate decarboxylase, which acts on pyruvate to form acetaldehyde and carbon dioxide. This enzyme is present in yeast and some bacteria but is not thought to be present in mammalian cells. The enzyme is known to be useful in the production of ethanol by fermentation. All mammalian cells contain pyruvate as it is a central component of the Krebs cycle. As noted above, some cancer cells may have significantly elevated levels of pyruvate. It is preferred that the portion is an enzymatically active portion of yeast, preferably Saccharomyces pyruvate decarboxylase, or more preferably an enzymatically active portion of Zymomonas pyruvate decarboxylase, which has advantageous higher stability and higher specific activity than other pyruvate decarboxylase enzymes (Konig (1998) “Subunit structure, function and organisation of pyruvate decarboxylases from various organisms” Biochim Biophys Acta 1385, 271-286).

By a “microsomal oxidase” is meant any enzyme found within the microsome or endoplasmic reticulum of a mammalian cell that is capable of oxidising an alcohol to an aldehyde. Other enzymes may be suitable, such as carbonyl reductase family enzymes, or dihydrodiol dehydrogenases (steroid dehydrogenases), which are involved (as is ADH) in drug metabolism in liver and in blood.

Isoforms of alcohol dehydrogenase and pyruvate decarboxylase may be preferred because of their high specific activity.

Singh (1995) Mutation Res. 337, 9-17 showed that acetaldehyde can be toxic and produce irreparable DNA damage, for example at a concentration of 1.56 mM. Thus an acetaldehyde concentration of, for example, about 1.6 mM may lead to directly toxic actions and/or to enhanced sensitivity to chemotherapeutic drugs. The acetaldehyde concentration, for example in serum, can be measured by high performance liquid chromatography (Lucas (1986) J Chromatography 382, 57-66 or DiPadova (1986) Alcohol Clin Exp Res 10, 86-89). The local concentration required for toxicity may be measured by in vitro cellular cytotoxicity assays known to those skilled in the art for example as described in Example 5. It is preferred that the local concentration of acetaldehyde produced is equal to or greater than that found to be toxic in an in vitro cellular cytotoxicity assay, for example as described in Example 5.

It will be appreciated that the enzyme substrate may be an endogenous substance (i.e. that it is normally present in the vicinity of the targeted cells at a concentration sufficient to be acted upon by the portion capable of converting a substrate to acetaldehyde to generate an effective amount of acetaldehyde) but it may also be an exogenous substrate (as defined previously) that may be acted upon to give rise to an elevated level of acetaldehyde. It is preferred that the enzyme substrate is an exogenous substance; it is particularly preferred that the substrate is ethanol.

The physiological metabolism of alcohol (ethanol) takes place in two stages as shown:
Stage 1: (Ethanol) C₂H₅OH+β-NAD→(Acetaldehyde) CH₃CHO+β-NADH

This reaction takes place primarily within liver cells. It is catalysed mainly (80+%) by the enzyme alcohol dehydrogenase (ADH) which is found in the cytosol of these cells. Two other enzyme systems also oxidise alcohol to acetaldehyde (−20%); they are catalase and the microsomal oxidases. This description is merely a suggestion of how alcohol dehydrogenase functions and is not to be taken as a limitation upon the scope of the invention.
Stage 2: (Acetaldehyde) CH₃CHO+β-NAD→(acetic acid ) CH₃COOH+β-NADH

This reaction also occurs within liver cells. This reaction is catalysed by aldehyde dehydrogenase 1 (ALDH1) and normally proceeds at a rate sufficient to prevent the build up of acetaldehyde. If the breakdown of acetaldehyde is inhibited by either the presence of a defective gene (common in Orientals) or by the therapeutic use of an enzyme inhibitor (Disulfiram “Antabuse”; a reversible inhibitor of ALDH1) then the build up of acetaldehyde leads to systemic toxicity. The acetic acid may be further converted to acetyl-CoA.

ALDH1 may be found in the cytosol of cells of most tissues, but is only found in significant amounts in the liver. The Km of this enzyme is low (22 μM) and it is responsible for the removal of acetaldehyde produced in the liver following ethanol ingestion.

Acetaldehyde dehydrogenase 2 (ALDH2) is a mitochondrial enzyme and is found in all nucleated cells, but not in erythrocytes. It also catalyses the oxidation of acetaldehyde, to acetic acid. It is not significantly inhibited by Disulfiram and has a lower Km (3.5 μM) for acetaldehyde than 25 ALDH1.

There are a number of other recently documented Aldehyde dehydrogenases; 3, 4, γ, 2a and 2b, but these are not thought to play an important role in acetaldehyde metabolism (Yoshida (1991) Prog Nucl Ac Res and Mol Biol 40, 255-287).

The administration of ethanol systemically by either oral intake or intravenous injection will allow the reaction stage I to occur in the tumour environment but not in the vicinity of cells not containing significant levels of the enzyme, leading to the production of locally damaging concentrations of acetaldehyde.

Grafstrom (1994) Carcinogenesis 15, 985-990 shows that ethanol is a well recognised drug. Levels of 500-2500 mg/l are well tolerated. For comparison, the limit beyond which it is illegal to drive in the UK is 800 mg/I ie 17 mM.

It will be appreciated that it is preferred if the amount of ethanol administered to the patient is chosen so as to elevate the level of acetaldehyde at the site of the targeted cell (such as a tumour cell) to an effective level but not to lead to systemic elevation to a toxic level. It is preferred if ethanol is administered so that a blood concentration between 10 and 2500 mg/l, more preferably 100 to 2300 mg/l, still more preferably 200 to 2000 mg/l, yet more preferably 500 to 2000 mg/l is achieved.

Methods of measuring concentrations of ethanol and other alcohols in blood or other fluids are known to those skilled in the art, and may be applied to determine the quantity of substrate (alcohol) that is required to be administered to produce a given concentration of substrate in the vicinity of the target cells. It will be appreciated that the substrate may be administered in multiple doses and over a period that may extend to several days. It will be appreciated that the substrate may be administered whilst an effective amount of the acetaldehyde forming portion remains at the site of the target cells. This may be measured by the conversion of the substrate into acetaldehyde.

Depending on the presence/absence/level of activity of the enzyme (aldehyde dehydrogenase) that catalyses stage 2 in the targeted tumour tissue it will be appreciated that it may be desirable to combine the administration of ethanol with the administration of Disulfiram or other inhibitor of aldehyde dehydrogenase to block the intra-tumoral breakdown of acetaldehyde.

Depending upon the enzyme kinetics and the concentration of NAD+ in the extracellular fluid it may be necessary to administer additional NAD+ to increase the concentration in the extra-cellular fluid. This may be done safely as shown in Birkmayer (1993) Acta Neurol. Scand 146, 32-35. The concentration of NAD+ may be measured by known methods, as described in Bishop (1959) J Biol Chem 234, 1233, who determined the normal blood level of NAD+ to be 29.8±5.9 μM. The need to administer NAD+ may also be determined by measuring the concentrations of acetaldehyde produced. If the acetaldehyde producing portion uses NADP+ as a cofactor instead of NAD+ then NADP+ may be administered.

Human ADH consists of a dimeric metallo-enzyme with five separate subclasses. Classes I, II and III are reviewed in (Jornvall (1987) Enzyme 37, 5-18. Each enzyme has 2 subunits each of 374 amino acids. The ADH genes have been cloned, sequenced and expressed recombinantly, as described in the following papers:
ADH I Ikuta (198S) PNAS USA 82, 2703-2707
ADH II Hoog (1987) Biochemistry 26, 1926-1932
ADH III Giri (1989) Biochem Biophys Res Comm 164, 453-460
ADH IV Yokayama (1994) Biochem Biophys Res Comm 203, 219-224
ADH V Ikuta (1986) PNAS USA 83, 634-638

These enzymes may all be made recombinantly and ADH may be purified from human liver, as reviewed by Jornvall (above). Purified horse liver ADH (product A6128) and yeast ADH (product A3263) are available from Sigma Pharmaceuticals and may be used, for example for in vitro investigations.

It is preferred that the enzyme used has a high ethanol to acetaldehyde activity but minimal activity for the conversion of acetaldehyde to acetic acid. The atypical human liver (class I) enzyme consisting of a B₂B₂ homodimer may be particularly useful as it has an 80-fold higher activity than the more usual B₁B₁ homodimer (Yoshida J Biol Chem 256, 12430-12436).

By enzymatically active portion of an enzyme, for example alcohol dehydrogenase, catalase, a microsomal oxidase or pyruvate decarboxylase, is included any variant, fragment, derivative or fusion of the enzyme, or any fusion of a variant, fragment or derivative of the enzyme, that retains the catalytic activity of the enzyme. It is particularly preferred, although not essential, that the variant or fragment or derivative or fusion of the said enzyme, or the fusion of the variant or fragment or derivative has at least 30% of the enzyme activity of a human said enzyme, or for pyruvate decarboxylase, of pyruvate decarboxylase from Zymomonas. For example, it is particularly preferred, although not essential, that the variant or fragment or derivative or fusion of the said alcohol dehydrogenase, or the fusion of the variant or fragment or derivative has at least 30% of the enzyme activity of a human alcohol dehydrogenase (for example B₁B₁ homodimer—see above). It is more preferred if the variant or fragment or derivative or fusion of the said enzyme, for example alcohol dehydrogenase, or the fusion of the variant or fragment or derivative has at least 50%, preferably at least 70% and more preferably at least 90% of the enzyme activity of a human said enzyme, for example human alcohol dehydrogenase, or for pyruvate decarboxylase, of pyruvate decarboxylase from Zymomonas.

It will be appreciated that the term “enzymatically active portion” includes the full length naturally occurring enzyme.

It is preferred that the alcohol dehydrogenase is not horse, mouse or rat alcohol dehydrogenase.

Variants (whether naturally-occurring or otherwise) may be made using the methods of protein engineering and site-directed mutagenesis well known in the art using the recombinant polynucleotides described below.

“Variants” of the polypeptide include insertions, deletions and substitutions, either conservative or non-conservative. By “conservative substitutions” is intended substituion of an amino acid residue with a chemically similar residue. Chemically similar residues may be grouped for example Gly, Ala; Val, Ile, Leu; Asp, Glu; Asn, Gln; Ser, Thr; Lys, Arg; and Phe, Tyr. Conservative substitutions/chemically similar amino acid groupings are well known in the art. Preferably substitutions are conservative, and preferably preserving enzymatic activity.

“Fusion” includes said enzyme, for example alcohol dehydrogenase, fused to any other polypeptide. For example, the said enzyme may be fused to a polypeptide such as glutathione-S-transferase (GST) or protein A in order to facilitate purification of said enzyme. Similarly, the said enzyme may be fused to an oligo-histidine tag such as His₆ or, less preferably, to an epitope recognised by an antibody such as the well known Myc tag epitope. Preferably, the enyzme, for example alcohol dehydrogenase, is not fused to a polypeptide that is known to be antigenic in humans.

Low levels of ADH activity may be detected in most tissues apart from the brain. However, with the exception of the liver, the level of expression is very low (Estonius (1996) FEBS Lett 397, 338-342; Engelhard (1993) Biochem Biophys Res Comm 193, 47-53). It is not found in the extracellular fluid that surrounds tissue cells.

The action of the enzyme when present in, at or near selected cells will be to catalyse the production of acetaldehyde, for example from the oxidation of ethanol or decarboxylation of pyruvate. This will lead to an elevation of the concentration of acetaldehyde in or in the micro-environment of selected cells. The elevation in the concentration of acetaldehyde may of itself prove damaging or preferably fatal to selected cells. The elevation in concentration of acetaldehyde may also be exploited to kill selected cells in conjunction with conventional cytotoxic agents including chemotherapeutic drugs and radiation therapy (Hahn (1983) Cancer Res. 43, 5789-5791).

The elevation in acetaldehyde concentration may also be exploited to kill selected cells by rendering them more immunogenic and therefore better able to elicit an immune response or to be damaged by an immune response. Enhanced immunogenicity of cells exposed to acetaldehyde has been shown in Kolber (1991) Alcohol-Alcohol 1, 277-280 and Terabayashi (1990) Alcohol Clin. Exp. Res. 14, 893-899, which concerns increased immunogenicity as witnessed by an appreciable T cell response to acetaldehyde-treated cells. Enhanced immunogenicity of cells exposed to alcohol with the inhibition of acetaldehyde breakdown is shown in Crossley (1996) Gut 27, 186-189. The immune response to acetaldehyde modified tumour cells may be further enhanced, for example by the administration of cytokines including but not limited to; G-CSF, GM-CSF, IL2, IL12 and interferons. Other approaches include the use of dendritic cells to enhance antigen presentation and the use of cytotoxic T cells that recognise the altered cells.

By “target cell-specific portion” is included any moiety that is effective in delivering the portion capable of converting a substrate to acetaldehyde preferentially to (including to the vicinity of or surface of) the target cell. Thus, the target cell-specific portion may be a moiety that results in the compound being retained preferentially in a tumour, or other location in which the target cells are found, for example as a result of the physical properties (for example size or molecular weight) of the target cell-specific portion/compound.

The target cell-specific portion of the molecule may recognise any suitable entity which is expressed by tumour cells, virally-infected cells, pathogenic micro-organisms, cells introduced as part of gene therapy or normal cells of the body which may require to be destroyed or damaged for a particular reason, for example cells involved in an autoimmune response, particularly cells of the immune system, more particularly cells that mediate an immune response to an autoantigen. It may be a cell surface entity, including, but not limited to, a tumour-associated antigen or a cell surface receptor. Preferably the cell surface entity is a tumour-associated antigen or cell surface receptor. Preferably the cell surface entity is a cell surface receptor. The entity must be present or accessible to the targeting portion in significantly greater concentrations in or on cells which are to be damaged or destroyed than in any normal tissue of the host that cannot be functionally replaced by other therapeutic means. The host is preferably a mammal, most preferably a human, but may be any vertebrate. It is preferred that the host is not a mouse or other rodent.

Tumour-associated antigens, when expressed on the cell membrane or released into tumour extracellular fluid are particularly suitable as targets for antibodies.

The term “tumour” will be understood to refer to all forms of neoplastic cell growth, including tumours of the lung, liver, blood cells (leukaemias), skin, pancreas, colon, prostate, uterus or breast.

Considerable work has already been carried out on antibodies and fragments thereof to tumour-associated antigens and antibodies or antibody fragments directed at carcinoembryonic antigen (CEA) and antibodies or their fragments directed at human chorionic gonadotrophin (hCG) can be conjugated to carboxypeptidase G2 and the resulting conjugate retains both antigen binding and catalytic function. Following intravenous injection of these conjugates they localise selectively in tumours expressing CEA or hCG respectively. Other antibodies are known to localise in tumours expressing the corresponding antigen. Such tumours may be primary and metastatic colorectal cancer (CEA) and choriocarcinoma (hCG) in human patients or other forms of cancer. Although such antibody-enzyme conjugates may also localise in some normal tissues expressing the respective antigens, antigen expression is more diffuse in normal tissues. Such antibody-enzyme conjugates may be bound to cell membranes via their respective antigens or trapped by antigen secreted into the interstitial space between cells.

Examples of tumour-associated, immune cell-associated and infection reagent-related antigens are given in Table 1.

Alternatively, the entity which is recognised may or may not be antigenic but can be recognised and selectively bound to in some other way. For example, it may be a characteristic cell surface receptor such as the receptor for melanocyte-stimulating hormone (MSH) which is expressed in high numbers in melanoma cells. The cell-specific portion may then be a compound or part thereof which specifically binds to the entity in a non-immune sense, for example as a substrate or analogue thereof for a cell-surface enzyme or as a messenger.

TABLE 1
Cell surface antigens for targeting

| | | |
|---|---|---|
| Antigen | Antibody | Existing uses |

a) Tumour Associated Antigens

| | | |
|---|---|---|
| Carcino-embryonic | C46 (Amersham) | Imaging and therapy of |
| Antigen | 85A12 (Unipath) | colon/rectum tumours. |
| Placental Alkaline | H17E2 (ICRF, | Imaging and therapy of |
| Phosphatase | Travers & Bodmer) | testicular and ovarian |
| | | cancers. |
| Pan Carcinoma | NR-LU-10 (NeoRx | Imaging and therapy of |
| | Corporation) | various carcinomas |
| | | including small cell |
| | | lung cancer |
| Polymorphic | HMFG1 (Taylor- | Imaging and therapy of |
| Epithelial | Papadimitriou, | ovarian cancer and |
| Mucin (Human | ICRE) | pleural effusions. |
| milk fat | | |
| globule) | | |
| B-human | W14 | Targeting of |
| Chorionic | | carboxypeptidase to |
| Gonadotropin | | human xenografi |
| | | choriocarcinoma in |
| | | nude mice (Searle et al |
| | | (1981) Br. J. Cancer |
| | | 44, 137-144). |
| A carbohydrate on | L6 (IgG2 a)¹ | Targeting of alkaline |
| Human | | phosphatase (Senter et |
| Carcinomas | | al (1988) PNAS USA |
| | | 85, 4842-4846. |
| CD2O Antigen on | iFS (IgG2a)² | Targeting of alkaline |
| B Lymphoma | | phosphatase (Senter et |
| (normal | | al (1988) PNAS USA |
| and neoplastic) | | 85, 4842-4846. |

Other antigens include alphafoetoprotein, Ca-125 and prostate
specific antigen.

b) Immune Cell Antigens

| | | |
|---|---|---|
| Pan T | OKT-3 (Ortho) | As anti-rejection therapy |
| Lymphocyte | | for kidney transplants. |
| Surface Antigen | | |
| (CD3) | | |
| B-lymphocyte | RFB4 (Janossy, | Immunotoxin therapy of B |
| Surface Antigen | Royal Free | cell lymphoma. |
| (CD22) | Hospital) | |
| Pan T | H65 (Bodmer and | Immunotoxin treatment of |
| Lymphocyte | Knowles, ICRF; | acute graft versus host |
| Surface Antigen | licensed t Xoma | disease, rheumatoid |
| (CD5) | Corp, USA) | arthritis. |

c) Infectious Agent-Related Antigens

| | | |
|---|---|---|
| Mumps virus- | Anti-mumps | Antibody conjugated to |
| related | polyclonal | diphtheria toxin for |
| | antibody | treatment of mumps |
| Hepatitis B | Anti HBs Ag | Immunotoxin against |
| Surface Antigen | | hepatoma |

¹ Hellström et al (1986) Cancer Res. 46, 3917-3923
² Clarke et al (1985) Proc. Natl. A cad. Sci. USA 82, 1766-1770

TABLE 2
Binding moieties for tumour-selective targets and tumour-associated
antigens

| Target | Binding moiety | Disease |
|---|---|---|
| Truncated EGFR | anti-EGFR mAb | Gliomas |
| Idiotypes | anti-id mAbs | B-cell lymphomas |
| EGFR (c-erbB1) | EGE, TGF″ anti- | Breast cancer |
| | EGFR mAb | |
| c-erbB2 | Mabs | Breast cancer |
| IL-2 receptor | IL-2 anti-Tac | Lymphoma and |
| | mAb | leukaemias |
| IL-4 receptor | IL-4 | Lymphoma and |
| | | leukaemias |
| IL-6 receptor | IL-6 | Lymphoma and |
| | | leukaemias |
| MSH | α-MSH | Melanomas |
| (melanocyte- | | |
| stimulating | | |
| hormone) | | |
| receptor | | |
| Transferrin | Transferrin anti- | Gliomas |
| receptor (TR) | TR mAb | |
| gp95/gp97 | mAbs | Melanomas |
| p-glycoprotein | mAbs | Drug-resistant |
| cells | | |
| cluster-1 antigen | mAbs | Small cell lung |
| (N-CAM) | | carcinomas |
| cluster-w4 | mAbs | Small cell lung |
| | | carcinomas |
| cluster-5A | mAbs | Small cell lung |
| | | carcinomas |
| cluster-6 (LeY) | mAbs | Small cell lung |
| | | carcinomas |
| PLAP (placental | mAbs | Some seminomas |
| alkaline | | Some ovarian; some non |
| phosphatase) | | small cell lung cancer |
| CA-125 | mAbs | Lung, ovarian |
| ESA (epithelial | mAbs | Carcinoma |
| specific antigen) | | |
| CD 19, 22, 37 | mAbs | B-cell lymphomas |
| 250 kDa | mAbs | Melanoma |
| Proteoglycan | mAbs | Breast cancer |
| p55 | | |
| TCR-IgH fusion | mAbs | Childhook T-cell |
| | | leukaemia |
| Blood gp A | mAbs | Gastric and colon tumours |
| antigen (in B or | | |
| O individuals) | | |
| Mucin protein | mAbs | Breast cancer |
| core | | |

The target cell-specific portion may be an entire antibody (usually, for convenience and specificity, a monoclonal antibody), a part or parts thereof (for example an F_{ab} fragment or F(ab′)₂) or a synthetic antibody or part thereof A conjugate comprising only part of an antibody may be advantageous by virtue of being cleared from the blood more quickly and may be less likely to undergo non-specific binding due to the F_{c} part. Suitable monoclonal antibodies to selected antigens may be prepared by known techniques, for example those disclosed in “Monoclonal Antibodies: A manual of techniques”, H. Zola (CRC Press, 1988) and in “Monoclonal Hybridoma Antibodies: techniques and Applications”, J G R Hurrell (CRC Press, 1982). Bispecific antibodies may be prepared by cell fusion, by reassociation of monovalent fragments or by chemical cross-linking of whole antibodies, with one part of the resulting bispecific antibody being directed to the cell-specific antigen and the other to the portion capable of converting a substrate to an acetaldehyde. Methods for preparing bispecific antibodies are disclosed in Corvalen et al, (1987) Cancer Immunol. Immunother. 24, 127-132 and 133-137 and 138-143.

The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sci. USA 81, 68S1-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the VH and VL partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

By “ScFv molecules” I mean molecules wherein the V_{H} and N_{L} partner domains are linked via a flexible oligopeptide.

The advantages of using antibody fragments, rather than whole antibodies, are several-fold. The smaller size of the fragments may lead to improved pharmacological properties, such as better penetration of solid tissue. Effector functions of whole antibodies, such as complement binding, are removed. Fab, Fv, ScFv and dAb antibody fragments can all be expressed in and secreted from E. coli, thus allowing the facile production of large amounts of the said fragments.

Whole antibodies, and F(ab′)₂ fragments are “bivalent”. By “bivalent” I mean that the said antibodies and F(ab′)₂ fragments have two antigen combining sites. In contrast, Fab, Fv, ScFv and dAb fragments are monovalent, having only one antigen combining sites. Fragmentation of intact immunoglobulins to produce F(ab′)₂ fragments is disclosed by Harwood et al (1985) Eur. J. Cancer Clin. Oncol. 21, 1515-1522.

IgG class antibodies are preferred.

It is preferred if the target cell-specific portion comprises an antibody or fragment or derivative thereof.

The target cell-specific portion may, however, be any compound which leads to the accumulation of the portion capable of converting a substrate to acetaldehyde at the site of the target cell (such as a tumour). For example, non-specific uptake of a molecule by a tumour may allow an adequate ratio of tumour-associated cytotoxic drug to non-tumour associated drug to be achieved, for example if the enzyme conjugate is cleared or inhibited when away from the tumour. Examples include the preferential uptake by tumour cells of liposomes and other macromolecules (Sands (1988) Cancer Res. 48, 188-193). Alterations to the blood supply to solid tumours may assist in the non-specific uptake of a macromolecule by a tumour.

The linking of antibody or other polypeptide to a protein (e.g. enzyme) portion capable of converting a substrate to acetaldehyde can be achieved by any convenient conventional method; e.g. chemical conjugation, biotinstreptavidin interactions or the production of a recombinant fusion protein.

It will be appreciated that the target-cell specific portion and the portion capable of converting a substrate to acetaldehyde may be linked by a “lock and key” system. Thus the target cell specific portion may further comprise a “lock” component and the portion capable of converting a substrate to acetaldehyde may further comprise a “key” component that interacts specifically and with high affinity with the “lock” component. An example is that the “lock” may be streptavidin or avidin and the “key” may be biotin, or vice versa. It will be appreciated that the system is not limited to streptavidin/biotin linking. It will also be appreciated that further “adapter” molecules may mediate the binding between the “lock” component and the “key” component. For example the target-cell specific portion and the portion capable of converting a substrate to an aldehyde may both further comprise biotin, and the linking of the two portions may be achieved by administration of free streptavidin/avidin. This forms a sandwich system (valency of biotin=1, valency of streptavidin/avidin=4). It will be appreciated that when an “adapter” molecule is used that the “lock” and “key” components may be the same or different types of molecule.

It will further be appreciated that the interaction of the “lock”, “key” and “adapter” (if used) components may take place in the body of a patient to which the target-cell specific portion and portion capable of converting a substrate to acetaldehyde are administered.

Thus an aspect of the invention is a system for targeting a portion capable of converting a substrate to acetaldehyde to a target cell comprising 1) a target-cell specific portion further comprising a “lock” component and 2) a portion capable of converting a substrate to acetaldehyde further comprising a “key” component that interacts specifically and with high affinity with the “lock” component or with an “adapter” component that interacts specifically and with high affinity with both the “lock” and the “key” component. By “high affinity” is meant an interaction with a K_{d} of between 10⁻¹³ and 10⁻¹⁶ M. By “interacts specifically” is meant that the component interacts with at least 100-fold higher affinity (and preferably at least 500-fold, or at least 1000-fold, or at least 2000-fold higher affinity) with the intended binding component than with other molecules that may be encountered by the first said component when administered to a patient.

It will be appreciated that this system may be used with any of the target-cell specific portions and portions capable of converting a substrate to acetaldehyde described herein. It is preferred that the portion capable of converting a substrate to acetaldehyde is a directly active portion as defined above.

It is preferred if the “lock”, “key” and “adapter” molecules are biotin or streptavidin/avidin. Biotin and streptavidin/avidin bind to each other with extremely high affinity (Kd=10⁻¹⁵M). This binding is far stronger than many non-covalent interactions, for example antibody/antigen interactions. This extremely high affinity and long lasting binding ability can be exploited in targeting systems in vivo. For example a monoclonal antibody with tumour binding specificity linked to streptavidin can be administered to a patient and allowed to localise within the tumour. This can then be followed by the administration of a second molecule that has been linked to biotin. This will bind rapidly and durably to the antibody/streptavidin on the cancer cell, with rapid excretion of the excess second molecule that does not bind to the antibody/streptavidin. This approach can be further extended to give what is termed a 3-step approach. In this the antibody is linked to biotin, and administration is followed by free streptavidin/avidin, and then finally by the second molecule which is also linked to biotin. This forms a sandwich system (valency of biotin=1, valency of streptavidin/avidin=4). Both the straightforward 2-step system (Paganelli (1988) Int. J. Cancer 2, 121-125) and the 3-step system (Dosio (1993) J. Nuclear Biological Med. 37, 228-232) are well described in vitro and in patients.

A further aspect of the invention provides a compound comprising a portion capable of converting a substrate to acetaldehyde and a “lock” or “key” or “adapter”. The portion capable of converting a substrate to acetaldehyde, the “lock”, the “key” or the “adapter” are as defined previously.

The linking of the target-cell specific portion and the portion capable of converting a substrate to acetaldehyde may be covalent or non-covalent. If the portion capable of converting a substrate to acetaldehyde is a polypeptide, the polypeptide portions can be linked together by any of the conventional ways of cross-linking polypeptides, such as those generally described in O'Sullivan et al., (Anal Biochem 100, (1979), 108). For example, these portions could be linked chemically using a cross-linking agent such as the N-hydroxysuccinimide ester of iodoacetic acid (NHIA) or N-succinimidyl-3-(2-pyridyldithio)propionate (SPDP) which react with thiol groups. Such cross-linking may be facilitated by the introduction of specific amino acids into one or both of the portions, especially free cysteine residues (i.e. not involved in disulphide interactions) or free lysine residues (i.e. not involved in critical interactions via the primary amine group). This is achieved by recombinant DNA manipulations well known to those skilled in the art, as described above.

Preferably, the two portions of the compound as defined in relation to the first aspect of the invention may be produced as a fusion compound polypeptide by recombinant DNA techniques whereby a length of DNA comprises respective regions encoding the two portions of the compound of the invention either adjacent to one another or separated by a region encoding a linker peptide which does not destroy the desired properties of the compound. Conceivably, the two portions of the compound may overlap wholly or partly. Thus, in this embodiment, the compound is characterised in that the target cell specific portion and the enzymatically active portion are fused within a single protein. A DNA construct encoding such a compound may be expressed in a suitable host in known ways to produce the compound.

A further aspect of the invention provides a compound as defined in relation to the first aspect of the invention, wherein the portion or polypeptide capable of converting a substrate to acetaldehyde is an enzymatically active portion of catalase or a microsomal oxidase or pyruvate decarboxylase, or of human alcohol dehydrogenase, preferably alcohol dehydrogenase β2.

A further aspect of the invention provides a polynucleotide construct encoding a compound according to the invention.

A variety of methods have been developed to operably link polynucleotides, especially DNA, to vectors for example via complementary cohesive termini. For instance, complementary homopolymer tracts can be added to the DNA segment to be inserted to the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, generated by endonuclease restriction digestion as described earlier, is treated with bacteriophage T4 DNA polymerase or E. Coli DNA polymerase I, enzymes that remove protruding, 3′-single-stranded termini with their 3′-5′-exonucleolytic activities, and fill in recessed 3′-ends with their polymerizing activities.

The combination of these activities therefore generates blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and ligated to an expression vector that has been cleaved with an enzyme that produces termini compatible with those of the DNA segment.

Synthetic linkers containing a variety of restriction endonuclease sites are 20 commercially available from a number of sources including International Biotechnologies Inc, New Haven, Conn., USA.

A desirable way to modify the DNA encoding the polypeptide of the invention is to use the polymerase chain reaction as disclosed by Saiki et al (1988) Science 239, 487-491. This method may be used for introducing the DNA into a suitable vector, for example by engineering in suitable restriction sites, or it may be used to modify the DNA in other useful ways as is known in the art. In this method the DNA to be enzymatically amplified is flanked by two specific primers which themselves become incorporated into the amplified DNA. The said specific primers may contain restriction endonuclease recognition sites which can be used for cloning into expression vectors using methods known in the art.

The DNA (or in the case of retroviral vectors, RNA) is then expressed in a suitable host to produce a polypeptide comprising the compound of the invention. Thus, the DNA encoding the polypeptide constituting the compound of the invention may be used in accordance with known techniques, appropriately modified in view of the teachings contained herein, to construct an expression vector, which is then used to transform an appropriate host cell for the expression and production of the polypeptide of the invention. Such techniques include those disclosed in U.S. Pat. No. 4,440,859 issued 3 Apr. 1984 to Rutter et al, U.S. Pat. No. 4,530,901 issued 23 Jul. 1985 to Weissman, U.S. Pat. No. 4,582,800 issued 15 Apr. 1986 to Crowl, U.S. Pat. No. 4,677,063 issued 30 Jun. 1987 to Mark et al, U.S. Pat. No. 4,678,751 issued 7 Jul. 1987 to Goeddel, U.S. Pat. No. 4,704,362 issued 3 Nov. 1987 to Itakura et al, U.S. Pat. No. 4,710,463 issued 1 Dec. 1987 to Murray, U.S. Pat. No. 4,757,006 issued 12 Jul. 1988 to Toole, Jr. et al, U.S. Pat. No. 4,766,075 issued 23 Aug. 1988 to Goeddel et al and U.S. Pat. No. 4,810,648 issued 7 Mar. 1989 to Stalker, all of which are incorporated herein by reference.

The DNA (or in the case of retroviral vectors, RNA) encoding the polypeptide constituting the compound of the invention may be joined to a wide variety of other DNA sequences for introduction into an appropriate host. The companion DNA will depend upon the nature of the host, the manner of the introduction of the DNA into the host, and whether episomal maintenance or integration is desired.

Generally, the DNA is inserted into an expression vector, such as a plasmid, in proper orientation and correct reading frame for expression. If necessary, the DNA may be linked to the appropriate transcriptional and translational regulatory control nucleotide sequences recognised by the desired host, although such controls are generally available in the expression vector. The vector is then introduced into the host through standard techniques. Generally, not all of the hosts will be transformed by the vector. Therefore, it will be necessary to select for transformed host cells. One selection technique involves incorporating into the expression vector a DNA sequence, with any necessary control elements, that codes for a selectable trait in the transformed cell, such as antibiotic resistance. Alternatively, the gene for such selectable trait can be on another vector, which is used to co-transform the desired host cell.

Host cells that have been transformed by the recombinant DNA of the invention are then cultured for a sufficient time and under appropriate conditions known to those skilled in the art in view of the teachings disclosed herein to permit the expression of the polypeptide, which can then be recovered.

Many expression systems are known, including bacteria (for example E. coli and Bacillus subtilis), yeasts (for example Saccharomyces cerevisiae), filamentous fungi (for example Aspergillus), plant cells, animal cells and insect cells.

The vectors include a prokaryotic replicon, such as the ColE1 ori, for propagation in a prokaryote, even if the vector is to be used for expression in other, non-prokaryotic, cell types. The vectors can also include an appropriate promoter such as a prokaryotic promoter capable of directing the expression (transcription and translation) of the genes in a bacterial host cell, such as E. coli transformed therewith.

A promoter is an expression control element formed by a DNA sequence 5 that permits binding of RNA polymerase and transcription to occur. Promoter sequences compatible with exemplary bacterial hosts are typically provided in plasmid vectors containing convenient restriction sites for insertion of a DNA segment of the present invention.

Typical prokaryotic vector plasmids are pUC18, pUC19, pBR322 and pBR329 available from Biorad Laboratories, (Richmond, Calif., USA) and pTrc99A and pKK223-3 available from Pharmacia, Piscataway, N.J., USA.

A typical mammalian cell vector plasmid is pSVL available from Pharmacia, Piscataway, N.J., USA. This vector uses the SV40 late promoter to drive expression of cloned genes, the highest level of expression being found in T antigen-producing cells, such as COS-1 cells.

An example of an inducible mammalian expression vector is pMSG, also available from Pharmacia. This vector uses the glucocorticoid-inducible promoter of the mouse mammary tumour virus long terminal repeat to drive expression of the cloned gene.

Useful yeast plasmid vectors are pRS403-406 and pRS413-416 and are generally available from Stratagene Cloning Systems, La Jolla, Calif. 92037, USA. Plasmids pRS403, pRS404, pRS405 and pRS406 are Yeast Integrating plasmids (YIps) and incorporate the yeast selectable markers H153, TRP1, LEU2 and URA3. Plasmids pRS413-416 are Yeast Centromere plasmids (YCps).

The present invention also relates to a host cell transformed with a polynucleotide vector construct of the present invention. The host cell can be either prokaryotic or eukaryotic. Bacterial cells are preferred prokaryotic host cells and typically are a strain of E. coli such as, for example, the E. coli strains DH5 available from Bethesda Research Laboratories Inc., Bethesda, Md., USA, and RR1 available from the American Type Culture Collection (ATCC) of Rockville, Md., USA (No ATCC 31343). Preferred eukaryotic host cells include yeast, insect and 10 mammalian cells, preferably vertebrate cells such as those from a mouse, rat, monkey or human fibroblastic cell line. Yeast host cells include YPH499, YPH500 and YPH501 which are generally available from Stratagene Cloning Systems, La Jolla, Calif. 92037, USA. Preferred mammalian host cells include Chinese hamster ovary (CHO) cells available from the ATCC as CCL6 1, NIH Swiss mouse embryo cells NIH/3T3 available from the ATCC as CRL 1658, and monkey kidney-derived COS-1 cells available from the ATCC as CRL 1650. Preferred insect cells are Sf9 cells which can be transfected with baculovirus expression vectors.

Transformation of appropriate cell hosts with a DNA construct of the present invention is accomplished by well known methods that typically depend on the type of vector used. With regard to transformation of prokaryotic host cells, see, for example, Cohen et al (1972) Proc. Natl. Acad. Sci. USA 69, 2110 and Sambrook et al (1989) Molecular Cloning, A Laboratory Manual, Cold Spring Harbor Laboratory, Cold Spring Harbor, N.Y. Transformation of yeast cells is described in Sherman et al (1986) Methods In Yeast Genetics, A Laboratory Manual, Cold Spring Harbor, N.Y. The method of Beggs (1978) Nature 275, 104-109 is also useful.

With regard to vertebrate cells, reagents useful in transfecting such cells, for example calcium phosphate and DEAE-dextran or liposome formulations, are available from Stratagene Cloning Systems, or Life Technologies Inc., Gaithersburg, Md. 20877, USA.

Electroporation is also useful for transforming and/or transfecting cells and is well known in the art for transforming yeast cell, bacterial cells, insect cells and vertebrate cells.

For example, many bacterial species may be transformed by the methods described in Luchansky et al (1988) Mol. Microbiol. 2, 637-646 incorporated herein by reference. The greatest number of transformants may be recovered following electroporation of the DNA-cell mixture suspended in 2.5×PEB using 6250V per cm at 25:FD, but optimal conditions may vary depending on the cell types used.

Methods for transformation of yeast by electroporation are disclosed in Becker & Guarente (1990) Methods Enzymol. 194, 182.

Successfully transformed cells, i.e. cells that contain a DNA construct of the present invention, can be identified by well known techniques. For example, cells resulting from the introduction of an expression construct of the present invention can be grown to produce the polypeptide of the invention, Cells can be harvested and lysed and their DNA content examined for the presence of the DNA using a method such as that described by Southern (1975) J. Mol. Biol. 98, 503 or Berent et al (1985) Biotech, 3, 208. Alternatively, the presence of the protein in the supernatant can be detected using antibodies as described below.

In addition to directly assaying for the presence of recombinant DNA, successful transformation can be confirmed by well known immunological methods when the recombinant DNA is capable of directing the expression of the protein. For example, cells successfully transformed with an expression vector produce proteins displaying appropriate antigenicity. Samples of cells suspected of being transformed are harvested and assayed for the protein using suitable antibodies.

Thus, in addition to the transformed host cells themselves, the present invention also contemplates a culture of those cells, preferably a monoclonal (clonally homogeneous) culture, or a culture derived from a monoclonal culture, in a nutrient medium.

In a further aspect of the invention, the target-cell specific portion may comprise a liposome. Liposomes are lipid spheres that have been used to selectively deliver agents including chemotherapy, drugs, radiation, antibodies and DNA to selected cells, including tumour cells (Weiner (1994) Immunomethods 4, 201-209; Sells (1995) Biotechniques 19, 72-76). The ability of liposomes to accumulate preferentially within areas of cancer cells can be used to deliver the portion capable of converting a substrate to acetaldehyde to the micro-environment of these cells where it may increase the concentration of acetaldehyde in the microenvironment. An example is that the enzyme alcohol dehydrogenase may be incorporated with liposomes, using known methods for the preparation of liposomes (Weiner (1994) Immunomethods 4, 201-209).

By “polynucleotide encoding a polypeptide capable of converting a substrate to acetaldehyde”, are included any such polynucleotide. The polynucleotide may be RNA or DNA; preferably it is DNA. The polynucleotide may encode, for example, an enzymatically active portion of alcohol dehydrogenase or pyruvate decarboxylase. The meaning of this term and the preferences for the polypeptide encoded by the polynucleotide are as defined in the description of the portion capable of converting a substrate to acetaldehyde given earlier. The said polynucleotide may be expressed within the selected cells, allowing the production of the polypeptide capable of converting a substrate to acetaldehyde within these cells. For example, a polynucleotide encoding an enzymatically active portion of alcohol dehydrogenase may be expressed in selected cells, for example tumour cells. The effect of expression of the portion capable of converting a substrate to acetaldehyde, for example alcohol dehydrogenase, within the selected cells may be to elevate the concentration of aldehyde with similar detrimental effects to those seen with enzyme activity in the extracellular fluid of the cellular micro-environment. The portion capable of converting a substrate to acetaldehyde may be expressed in such a way that it is exported from the cell, for example it may be expressed as a fusion protein with a signal peptide. Examples of suitable signal sequences are well known in the art.

It will be understood that when the inactivating portion comprises a polynucleotide encoding a polypeptide capable of converting a substrate to acetaldehyde, the target cell-specific portion of the compound of the invention is one which is adapted to deliver the polynucleotide (genetic construct) to the target cell.

Preferably, the genetic construct is adapted for delivery to a cell, preferably a human cell. More preferably, the genetic construct is adapted for delivery to a cell in an animal body, more preferably a mammalian body; still more preferably it is adapted for delivery to a cell in a human body. Most preferably, the genetic construct is suitable for carrying out gene therapy, as well known to those skilled in the art.

Means and methods of introducing a genetic construct into a cell in an animal body are known in the art. For example, the constructs of the invention may be introduced into the tumour cells by any convenient method, for example methods involving retroviruses, so that the construct is inserted into the genome of the tumour cell. For example, in Kuriyama et al (1991) Cell Struc. and Func. 16, 503-510 purified retroviruses are administered. Retroviruses provide a potential means of selectively infecting cancer cells because they can only integrate into the genome of dividing cells; most normal cells surrounding cancers are in a quiescent, non-receptive stage of cell growth or, at least, are dividing much less rapidly than the tumour cells. Retroviral DNA constructs which contain a suitable promoter segment and a polynucleotide encoding a polypeptide capable of converting a substrate to acetaldehyde, for example alcohol dehydrogenase or a variant or fragment or fusion or derivative as defined may be made using methods well known in the art. To produce active retrovirus from such a construct it is usual to use an ecotropic psi2 packaging cell line grown in Dulbecco's modified Eagle's medium (DMEM) containing 10% foetal calf serum (FCS). Transfection of the cell line is conveniently by calcium phosphate co-precipitation, and stable transformants are selected by addition of G418 to a final concentration of 1 mg/ml (assuming the retroviral construct contains a neo^{R} gene). Independent colonies are isolated and expanded and the culture supernatant removed, filtered through a 0.45 μm pore-size filter and stored at −70° C. For the introduction of the retrovirus into the tumour cells, it is convenient to inject directly retroviral supernatant to which 10 μg/ml Polybrene has been added. For tumours exceeding 10 mm in diameter it is appropriate to inject between 0.1 ml and 1 ml of retroviral supernatant; preferably 0.5 ml.

Alternatively, as described in Culver et al (1992) Science 256, 1550-1552, cells which produce retroviruses are injected into the tumour. The retrovirus-producing cells so introduced are engineered to actively produce retroviral vector particles so that continuous productions of the vector occurred within the tumour mass in situ. Thus, proliferating tumour cells can be successfully transduced in vivo if mixed with retroviral vector-producing cells.

Targeted retroviruses are also available for use in the invention; for example, sequences conferring specific binding affinities may be engineered into preexisting viral env genes (see Miller & Vile (1995) Faseb J. 9, 190-199 for a review of this and other targeted vectors for gene therapy).

Other methods involve simple delivery of the construct into the cell for expression therein either for a limited time or, following integration into the genome, for a longer time. An example of the laffer approach includes (preferably tumour-cell-targeted) liposomes (Missander et al (1992) Cancer Res. 52, 646-653).

Immunoliposomes (antibody-directed liposomes) are especially useful in targeting to cancer cell types which over-express a cell surface protein for which antibodies are available (see Table 1 for examples). For the preparation of immuno-liposomes MPB-PE (N-[4-(p-maleimidophenyl)-butyryl]-phosphatidylethanolamine) is synthesised according to the method of Martin & Papahadjopoulos (1982) J. Biol. Chem. 257, 286-288. MPB-PE is incorporated into the liposomal bilayers to allow a covalent coupling of the antibody, or fragment thereof, to the liposomal surface. The liposome is conveniently loaded with the DNA or other genetic construct of the invention for delivery to the target cells, for example, by forming the said liposomes in a solution of the DNA or other genetic construct, followed by sequential extrusion through polycarbonate membrane filters with 0.6 μm and 0.2 μm pore size under nitrogen pressures up to 0.8 MPa. After extrusion, entrapped DNA construct is separated from free DNA construct by ultracentrifugation at 80 000×g for 45 min. Freshly prepared MPB-PE-liposomes in deoxygenated buffer are mixed with freshly prepared antibody (or fragment thereof) and the coupling reactions are carried out in a nitrogen atmosphere at 4° C. under constant end over end rotation overnight. The immunoliposomes are separated from unconjugated antibodies by ultracentrifugation at 80 000×g for 45 min Immunoliposomes may be injected intraperitoneally or directly into the tumour.

Other methods of delivery include adenoviruses carrying external DNA via an antibody-polylysine bridge (see Curiel Prog. Med. Virol. 40, 1-18) and transferrin-polycation conjugates as carriers (Wagner et al (1990) Proc. Natl. Acad. Sci. USA 87, 3410-3414). In the first of these methods a polycation-antibody complex is formed with the DNA construct or other genetic construct of the invention, wherein the antibody is specific for either wild-type adenovirus or a variant adenovirus in which a new epitope has been introduced which binds the antibody. The polycation moiety binds the DNA via electrostatic interactions with the phosphate backbone. It is preferred if the polycation is polylysine.

The DNA may also be delivered by adenovirus wherein it is present within the adenovirus particle, for example, as described below.

In the second of these methods, a high-efficiency nucleic acid delivery system that uses receptor-mediated endocytosis to carry DNA macromolecules into cells is employed. This is accomplished by conjugating the iron-transport protein transferrin to polycations that bind nucleic acids. Human transferrin, or the chicken homologue conalbumin, or combinations thereof is covalently linked to the small DNA-binding protein protamine or to polylysines of various sizes through a disulfide linkage. These modified transferring molecules maintain their ability to bind their cognate receptor and to mediate efficient iron transport into the cell. The transferrin-polycation molecules form electrophoretically stable complexes with DNA constructs or other genetic constructs of the invention independent of nucleic acid size (from short oligonucleotides to DNA of 21 kilobase pairs). When complexes of transferrin-polycation and the DNA constructs or other genetic constructs of the invention are supplied to the tumour cells, a high level of expression from the construct in the cells is expected.

High-efficiency receptor-mediated delivery of the DNA constructs or other genetic constructs of the invention using the endosome-disruption activity of defective or chemically inactivated adenovirus particles produced by the methods of Cotten et al (1992) Proc. Natl. Acad. Sci. USA 89, 6094-6098 may also be used. This approach appears to rely on the fact that adenoviruses are adapted to allow release of their DNA from an endosome without passage through the lysosome, and in the presence of, for example transferrin linked to the DNA construct or other genetic construct, the construct is taken up by the cell by the same route as the adenovirus particle.

This approach has the advantages that there is no need to use complex retroviral constructs; there is no permanent modification of the genome as occurs with retroviral infection; and the targeted expression system is coupled with a targeted delivery system, thus reducing toxicity to other cell types.

It may be desirable to locally perfuse a tumour with the suitable delivery vehicle comprising the genetic construct for a period of time; additionally or alternatively the delivery vehicle or genetic construct can be injected directly into accessible tumours.

It will be appreciated that “naked DNA” and DNA complexed with cationic and neutral lipids may also be useful in introducing the DNA of the invention into cells of the patient to be treated. Non-viral approaches to gene therapy are described in Ledley (1995) Human Gene Therapy 6, 1129-1144.

Thus, it will be appreciated that a further aspect of the invention provides a composition comprising a genetic construct as defined in relation to the invention and means for introducing said genetic construct into a cell, preferably the cell of an animal body.

Alternative targeted delivery systems are also known such as the modified adenovirus system described in WO 94/10323 wherein, typically, the DNA is carried within the adenovirus, or adenovirus-like, particle. Michael et al (1995) Gene Therapy 2, 660-668 describes modification of adenovirus to add a cell-selective moiety into a fibre protein. Mutant adenoviruses which replicate selectively in p53-deficient human tumour cells, such as those described in Bischoff et al (1996) Science 274, 373-376 are also useful for delivering the genetic construct of the invention to a cell. Thus, it will be appreciated that a further aspect of the invention provides a virus or virus-like particle comprising a genetic construct of the invention. Other suitable viruses or virus-like particles include HSV, AAV, vaccinia and parvovirus.

It will be appreciated that the polynucleotide need not be one which has a target cell-specific promoter to drive the expression of the polypeptide capable of converting a substrate to acetaldehyde since the compound comprises a target cell-specific portion as described above for targeting the polynucleotide to the target cell. However, it may be advantageous if the polynucleotide comprises a target cell-specific promoter operably linked to a polynucleotide encoding the polypeptide capable of converting a substrate to acetaldehyde.

It will be further appreciated that target cell-specific expression of the polypeptide capable of converting a substrate to acetaldehyde, for example an enzymatically active portion of alcohol dehydrogenase may be achieved using a polynucleotide or genetic construct comprising a target cell-specific promoter whether or not the polynucleotide or genetic construct is comprised in a compound comprising a target-cell specific portion and a portion capable of converting a substrate to acetaldehyde.

Thus, as noted above, a further aspect of the invention provides a compound comprising a recombinant polynucleotide comprising a target cell-specific promoter operably linked to a polynucleotide encoding a polypeptide capable of converting a substrate to acetaldehyde wherein the portion or polypeptide capable of converting a substrate to acetaldehyde is an enzymatically active portion of catalase or a microsomal oxidase or pyruvate decarboxylase, or of human alcohol dehydrogenase, preferably S alcohol dehydrogenase β2.

Preferably the target cell-specific promoter is a tumour cell-specific promoter.

Useful genetic elements which are target cell-specific promoters are given below but new ones are being discovered all of the time which will be useful in this embodiment of the invention.

The tyrosinase and TRP-1 genes both encode proteins which play key 15 roles in the synthesis of the pigment melanin, a specific product of melanocytic cells. The 5′ ends of the tyrosinase and tyrosinase-related protein (TRP-1) genes confer tissue specificity of expression on genes cloned downstream of these promoter elements.

The 5′ sequences of these genes are described in Bradl, M. et al (1991) Proc. Natl. Acad. Sci. USA 88, 164-168 and Jackson, I. J. et al (1991) Nucleic Acids Res. 19, 3799-3804.

Prostate-specific antigen (PSA) is one of the major protein constituents of 25 the human prostate secretion. It has become a useful marker for the detection and monitoring of prostate cancer. The gene encoding PSA and its promoter region which directs the prostate-specific expression of PSA have been described (Lundwall (1989) Biochem. Biophys. Res. Comm. 161, 1151-1159; Riegman et al (1989) Biochem. Biophys. Res. Comm. 159, 95-102; Brawer (1991) Acta Oncol. 30, 161-168).

Carcinoembryonic antigen (CEA) is a widely used tumour marker, 5 especially in the surveillance of colonic cancer patients. Although CEA is also present in some normal tissues, it is apparently expressed at higher levels in tumorous tissues than in corresponding normal tissues. The complete gene encoding CEA has been cloned and its promoter region analysed. A CEA gene promoter construct, containing approximately 400 nucleotides upstream from the translational start, showed nine times higher activity in the adenocarcinoma cell line 5W3 03, compared with the HeLa cell line. This indicates that cis-acting sequences which convey cell type specific expression are contained within this region (Schrewe et al (1990) Mol. Cell. Biol. 10, 2738-2748).

The mucin gene, MUC1, contains 5′ flanking sequences which are able to direct expression selectively in breast and pancreatic cell lines, but not in non-epithelial cell lines as taught in WO 91/09867.

The alpha-fetoprotein (AFP) enhancer may be useful to drive pancreatic tumour-selective expression (Su et al (1996) Hum. Gene Ther. 7, 463-470).

The genetic constructs of the invention can be prepared using methods 25 well known in the art.

It will be understood that a compound comprising a polynucleotide encoding a polypeptide capable of converting a substrate to acetaldehyde and a target cell specific promoter, may also comprise a target cell specific antibody or liposome.

It will be appreciated that the invention envisages any means by which an activity capable of converting a substrate to acetaldehyde may be selectively targeted to chosen cells, for example tumour cells.

If the compound of the invention or as defined in relation to the first aspect of the invention contains a polypeptide portion and a polynucleotide portion, the portions may be linked chemically using materials such as benzoquinone, as described by Poncet et al., (Gene Therapy, 3 (1996), 731-738), which is incorporated herein by reference. Alternatively, conjugation may be facilitated by the introduction of specific amino acids into the polypeptide portion and specific derivatised nucleotides into the nucleic acid portion. For example, one or more free cysteines introduced into the polypeptide by recombinant DNA manipulations well known to those skilled in the art, as described above, could be cross-linked to one or more free primary amino groups introduced into the nucleic acid portion by introduction of modified nucleotides either at the termini of the nucleic acid, for example by conversion of the 5′-phosphate group, or internally using nick translation, PCR or transcription reactions to introduce modified nucleotides, for example biotinylated nucleotides, available from Life Technologies.

It will be apparent that an elevation in acetaldehyde concentration caused by the acetaldehyde producing portion may have detrimental effects on all cells exposed to both the activity of said acetaldehyde producing portion and the necessary substrate. Whilst activity of the targeted portion capable of converting a substrate to acetaldehyde within the microenvironment of the selected cells may have therapeutic benefits by causing or promoting the death of these cells, an elevation of the level of acetaldehyde, via the actions of the acetaldehyde producing portion when present in the extracellular fluid around normal cells, may, under certain circumstances, cause unnecessary and potentially dangerous damage. Experience with targeting methods, including the antibody and liposomal delivery systems indicates that when initially administered these systems will diffuse (non-specifically) throughout the extracellular fluid. The selective targeting abilities of these methods only becomes apparent after a delay period during which there is clearance from the generality of the body, except those areas specifically targeted by the antibody or liposomal target cell specific delivery systems. For example, the delay periods commonly employed in the 2-step or 3-step biotin/streptavidin systems are 24 hours per step (Paganelli (1994) Eur J Nuclear Med. 21, 314-321 and Paganelli (1988)). The compounds or components of the system of the invention that do not bind to tissue cells will be removed from the body by natural clearance pathways, for example uptake by liver cells by phagocytosis. The removal may take a similar time to the delay periods employed using similar systems as described above.

It will be appreciated that use of a two or three step system, for example a system using biotin/streptavidin or biotin/streptavidin/biotin binding to link the target-cell specific portion and portion capable of converting a substrate to a acetaldehyde, as described above, is envisaged as an example of the above method. When such a method is used, it will be appreciated that the target-cell specific portion will be administered prior to administration of the “adapter” (in the case of a three-step system) and the portion capable of converting a substrate to acetaldehyde. It will further be appreciated that it is preferred that administration of the substrate commences after administration of the portion capable of converting a substrate to acetaldehyde.

It will be appreciated that the normal levels of a substrate of the portion capable of converting a substrate to acetaldehyde will preferably be low, such that toxic levels of the acetaldehyde are not produced prior to administration of the exogenous (as defined above) substrate. Methods of measuring the level of, for example, alcohols in fluids such as blood are known. It will be appreciated that these methods may be used to determine whether the patient has a level of alcohol present in the blood or other fluid that would render administration of the portion capable of converting a substrate to acetaldehyde undesirable, in that toxic amounts of acetaldehyde may be produced.

For example, after localisation of the enzyme alcohol dehydrogenase has occurred by antibody or liposomal delivery system and clearance from the generality of the extracellular fluid has occurred, the substrate (ethanol) may be administered. Clearance from the generality of the extracellular fluid may take about 24 hours.

The administration of alcohol systemically by either oral intake or intravenously will allow the reaction stage 1 to occur in the tumour environment but not in the environment of cells not containing the enzyme, leading to the production of locally damaging concentrations of acetaldehyde.

Depending on the presence/absence/level of activity of the enzymes (aldehyde dehydrogenases 1 and 2) that catalyse stage 2 in the targeted tumour tissue it may or may not be desirable to combine the administration of compound and (usually) substrate, for example ethanol, with the administration of an inhibitor of aldehyde dehydrogenase, for example Disulfiram which inhibits aldehyde dehydrogenase 1 (ALDH 1), to block the intra-tumoral breakdown of acetaldehyde. Disulfiram is available from Dumex Ltd, Tring Business Centre, Upper Icknield Way, Tring, Herts HP23 4JX. Side-effects of Disulfiram are generally rare and mild. They include drowsiness, fatigue, nausea and vomiting (ABPI Compendium of Data Sheets 1996-1007, page 277).

Depending upon the enzyme kinetic and the concentration of NAD⁺ in the extracellular fluid it may be necessary to administer additional NAD⁺ to increase the concentration in the extra-cellular fluid. This may be done safely as shown in Birkmayer (1996) Acta Neurol. Scand 146, 32-35. Thus NAD⁺ or other suitable cofactor may also be administered to the 15 host.

The desired result of this process is the selective delivery of elevation of acetaldehyde concentration to cells selected by the delivery system. It will readily be appreciated, for example, that the delivery of the enzyme alcohol dehydrogenase to specific cells, including tumour cells, according to this invention, can be used therapeutically.

The compounds or system of the invention or as defined in relation to the first aspect of the invention are administered in any suitable way, usually parenterally, for example intravenously, intraperitoneally or intravesically, in standard sterile, non-pyrogenic formulations of diluents and carriers. It will be understood that inhibitors of aldehyde dehydrogenase may be administered in any suitable way known in the art, for example orally or parenterally.

It will further be understood that a chemotherapeutic agent or a radiation therapy or other cytotoxic therapy may also be administered to the host. Elevation of acetaldehyde concentration may enhance the cell damage and death produced by such other therapies.

An immunosuppressive agent may also be administered to the host. This may help overcome the host response to foreign proteins. An immunosuppressive agent such as cyclosporin A may be used to delay such responses and allow treatment to continue for an extended period.

A further aspect of the invention comprises use of a compound or system of the invention or as defined in relation to the first aspect of the invention in the manufacture of a medicament for the treatment of a host with a condition in which target cells are beneficially destroyed. The condition may be cancer.

The invention also provides the use of human alcohol dehydrogenase or pyruvate decarboxylase or catalase or a microsomal oxidase or an enzymatically active portion thereof in medicine. The use of alcohol dehydrogenase or catalase or a microsomal oxidase or pyruvate decarboxylase or an enzymatically active portion thereof in the manufacture of a medicament for the treatment of cancer is also described.

Preferably the host is, has been, or will be administered a substrate that is converted to acetaldehyde by the portion capable of converting said substrate to acetaldehyde, and optionally a substance which is capable of inhibiting aldehyde dehydrogenase. Administration of the substrate may not start until the ratio of portion capable of converting a substrate to acetaldehyde bound to target cells to said portion not bound to the target cells has reached a desired value.

A further aspect of the invention comprises the use of ethanol (a substrate of alcohol dehydrogenase or catalase or a microsomal oxidase) or pyruvate (a substrate of pyruvate decarboxylase) in the manufacture of a medicament for use in the treatment of a host with a condition in which target cells are beneficially destroyed. The condition may be cancer. Preferably the host is, has been, or will be, also treated with a compound or system of the invention or as defined in relation to the first aspect of the invention, and optionally a substance which is capable of inhibiting aldehyde dehydrogenase.

A further aspect of the invention comprises a therapeutic system comprising a compound or system of the invention or as defined in relation to the first aspect of the invention, and a second component which is converted to acetaldehyde by the portion capable of converting a substrate to acetaldehyde, and optionally a third component that is capable of inhibiting aldehyde dehydrogenase. An example is such a system in which the portion capable of converting a substrate to acetaldehyde is alcohol dehydrogenase, the second component is ethanol, and the third component is Disulfiram.

A still further aspect is the use of a substance which is capable of inhibiting aldehyde dehydrogenase, for example Disulfiram, in the manufacture of a medicament for the treatment of a host with a condition in which target cells are beneficially destroyed. The condition may be cancer. Preferably the host is, has been, or will be, also treated with a compound or system of the invention or as defined in relation to the first aspect of the invention, and optionally a substrate that is converted to acetaldehyde by the portion capable of converting said substrate to acetaldehyde, for example ethanol or pyruvate.

Additional Aspects

It will be appreciated that the invention also embraces the following aspects.

A method as described above in which the portion capable of converting a substrate to acetaldehyde is directly active;

A method as described above wherein the portion capable of converting a substrate to acetaldehyde does so indirectly, in that it comprises a polynucleotide encoding a polypeptide capable of converting a substrate to acetaldehyde;

A method as described above in which the host has cancer.

Use of an inhibitor of aldehyde dehydrogenase in the manufacture of a medicament for use in the treatment of a host with a condition in which target cells are beneficially destroyed. Preferably the inhibitor is Disulfiram. Preferably the condition is cancer.

A compound comprising a portion capable of converting a substrate to acetaldehyde and a lock or key or adapter.

A system for targeting a portion capable of converting a substrate to acetaldehyde to a target cell as described above.

Use of a compound or system as described above in the manufacture of a medicament for the treatment of a host with a condition in which target cells are beneficially destroyed.

The present invention will now be described, by way of example, in more detail with reference to the following figures:

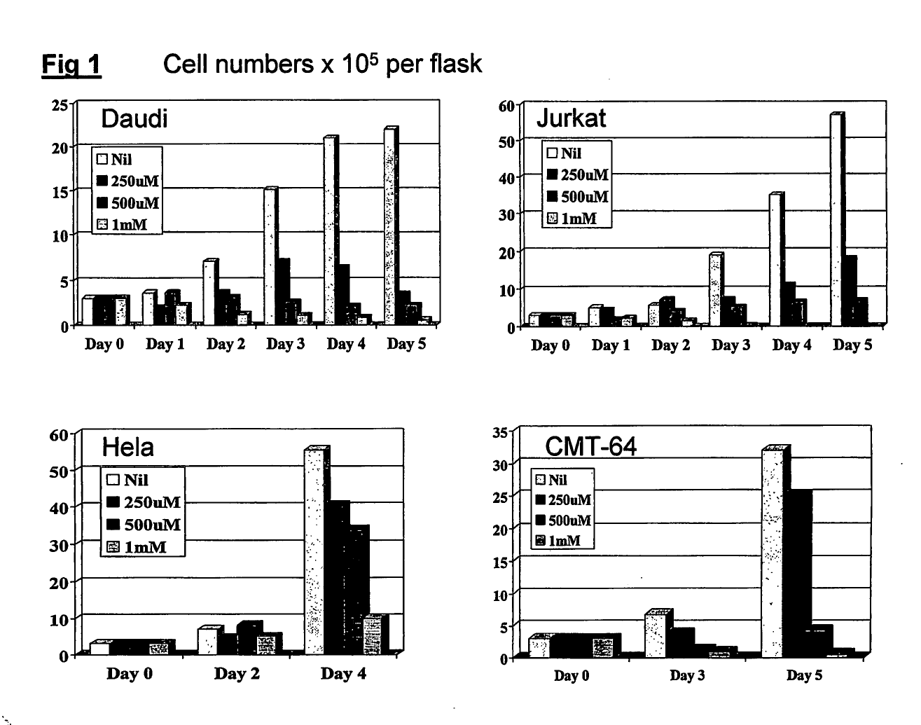

FIG. 1 which shows bar charts.

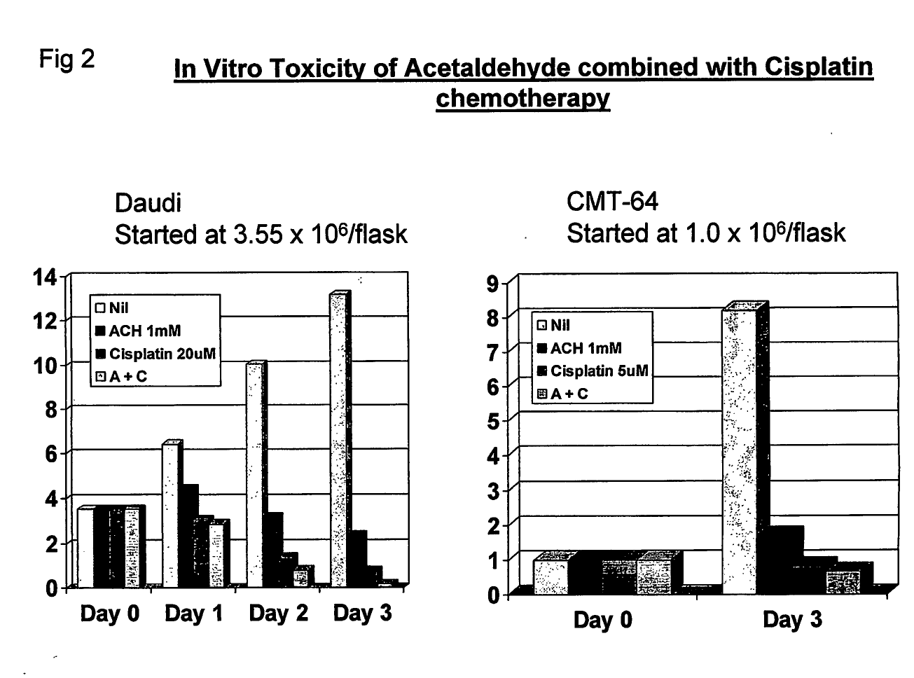

FIG. 2 which shows bar charts.

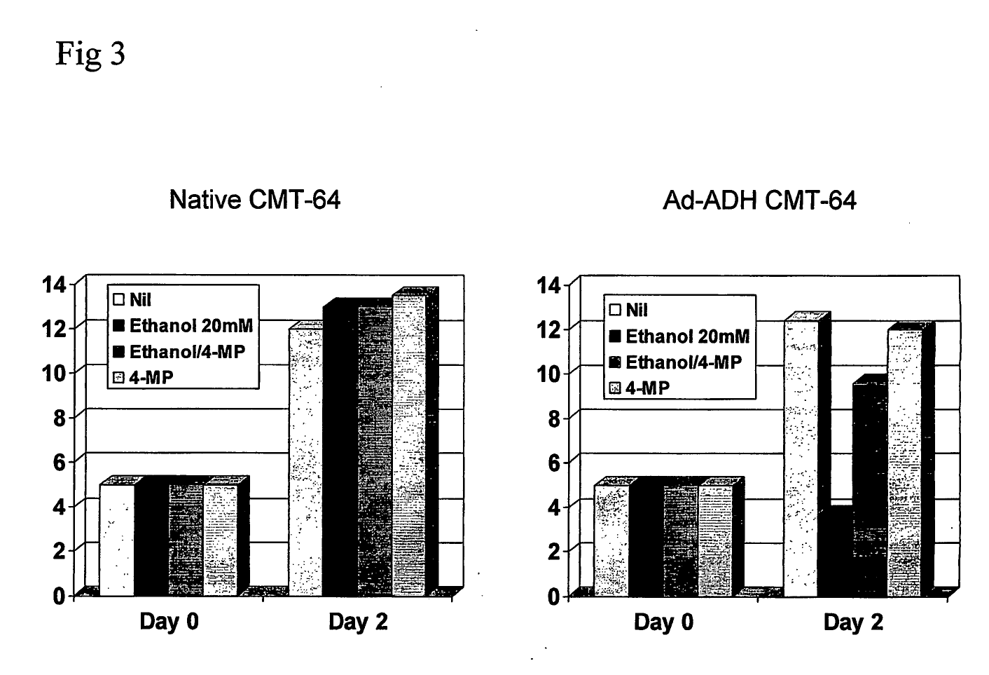

FIG. 3 which shows bar charts.

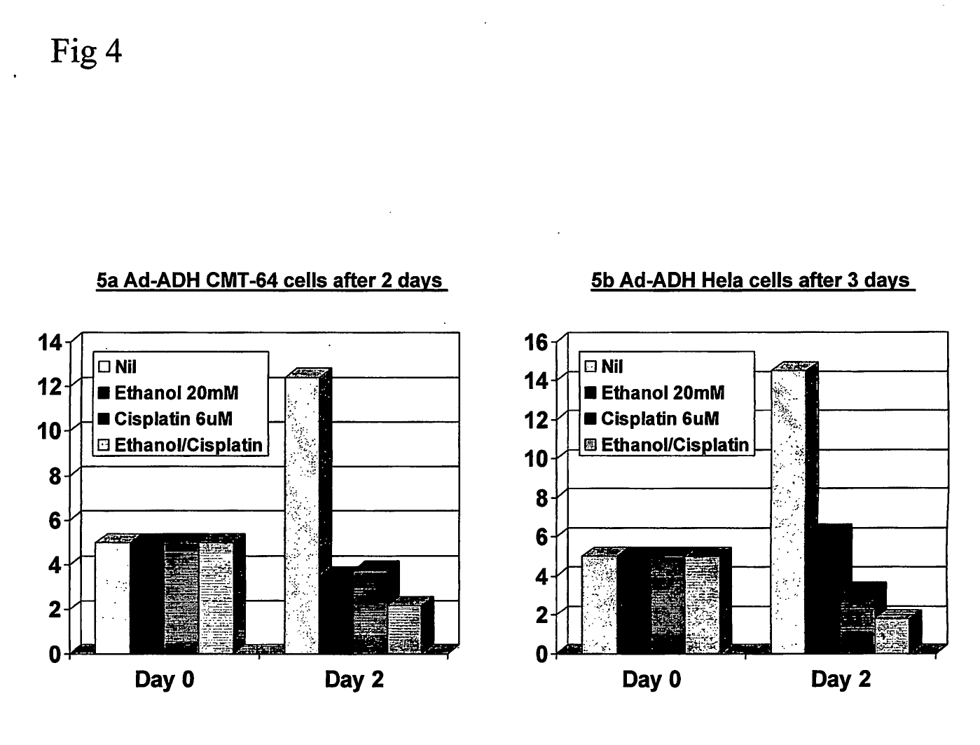

FIG. 4 which shows bar charts.

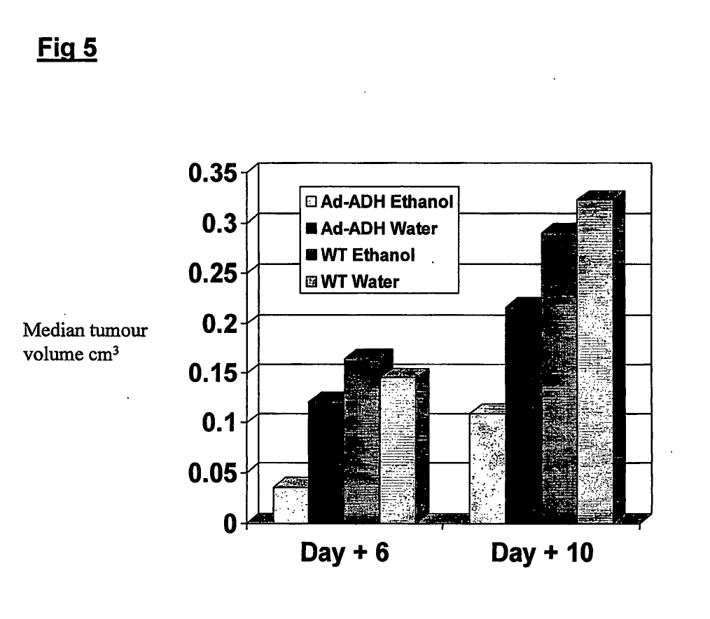

FIG. 5 which shows bar charts.

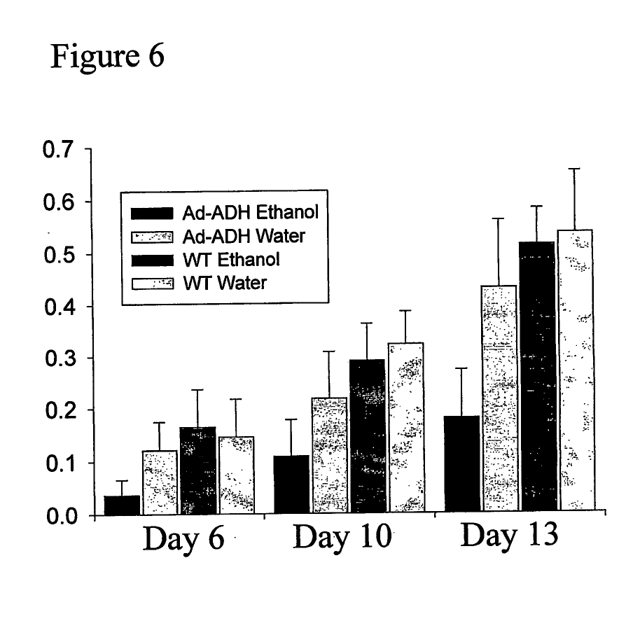

FIG. 6 which shows bar charts.

### EXAMPLE 1

### Preparation of an Alcohol Dehydrogenase—Antibody Compound

Recombinant human alcohol dehydrogenase is expressed in S. cerevisiae or E. coli from the cDNA sequence of Xu et al (1988) Genomics 2, 209-214 (ADH β2) or of Iknta (1985) PNAS 82, 5578-5578 and Iknta et al (1985) PNAS 82, 2703-2707 (ADH β31). Using a suitable expression system, which facilitates purification of the recombinant protein. It is purified using known methods appropriate to the vector system. Alternatively, purified horse alcohol dehydrogenase is purchased from Sigma (product A 6128).

An antibody (NR-LU-10; NeoRx Corporation) cross-reacting with the Pan Carcinoma antigen implicated in various carcinomas, including small cell lung cancer, is obtained and cross-linked to alcohol dehydrogenase obtained as above. The linking is carried out by methods described in O'Sullivan et al (1979). In particular, the linking is achieved by treatment with the N-hydroxysuccinimide ester of iodoacetic acid (NHIA).

### EXAMPLE 2

### Preparation of an Alcohol Dehydrogenase—Liposome Compound

Alcohol dehydrogenase is prepared as described in Example 1. It is incorporated into liposomes as described in Weiner (1994).

Immunoliposomes are prepared using N-[4(p-maleimidophenyl)-butyryl]-phosphatidylethanolamine) (MPB-PE), synthesised as described in Martin & Papahadjopoulos (1982). MPB-PE is incorporated into the liposomal bilayers to allow covalent coupling of the NR-LU-10 (NeoRx Corporation) antibody to the liposomes. Alcohol dehydrogenase prepared as described in Example 1 or DNA constructs prepared as described in Example 3 are incorporated into the liposomes, by forming the liposomes in a solution of alcohol dehydrogenase or the DNA construct, followed by sequential extension through polycarbonate membrane filters with 0.6 μm and 0.2 μm pore size under nitrogen pressures up to 0.8 MPa. After extrusion, entrapped DNA construct is separated from free DNA construct by ultracentrifugation at 80 000×g for 45 min. Freshly prepared MPBPE-liposomes in deoxygenated buffer are mixed with freshly prepared antibody (or fragment thereof) and the coupling reactions are carried out in a nitrogen atmosphere at 4° C. under constant end over end rotation overnight. The immunoliposomes are separated from unconjugated antibodies by ultracentrifugation at 80 000×g for 45 min.

### EXAMPLE 3

### Preparation of a DNA Construct Comprising a Sequence Encoding Alcohol Dehydrogenase, Controlled by a Tumour-cell Specific Promoter

The cDNA sequence encoding human alcohol dehydrogenase as described in Example 1 is joined to the promoter region of the gene encoding PSA (prostate specific antigen) (Lundwall (1989); Riegman et al (1989); Brawer (1991)). This construct may direct expression of alcohol dehydrogenase in prostate-derived cells. The construct is made by PCR based techniques and is verified by DNA sequencing using known techniques.

Alternatively, the cDNA encoding alcohol dehydrogenase is linked to the promoter region of the gene encoding CEA (carcinoembryogenic antigen) (Schrewe et al (1990)) which appears to be expressed at elevated levels in colonic cancer cells.

### EXAMPLE 4

### Administration of an Alcohol Dehydrogenase—Antibody Compound, Ethanol and an Inhibitor of Aldehyde Dehydrogenase (Disulfiram) to a Patient

An alcohol dehydrogenase—antibody compound prepared as described in Example 1 is administered to a patient with small cell lung cancer. After cessation of administration of the compound, tissue plasma samples are taken at intervals from a site distal to the lungs and sites of metastases and are analysed for the presence of elevated levels of alcohol dehydrogenase. When the level of alcohol dehydrogenase has decreased to a level not thought to be able to cause an elevation of aldehyde levels sufficient to damage cells, ethanol is administered orally to the patient to achieve a blood concentration of 1000 mg/L.

The treatment is performed on patients who are also undergoing chemotherapy and/or radiation therapy.

Elevation of the acetaldehyde concentration at the tumour site is monitored by extraction of tissue fluid and analysis by HPLC (high performance liquid chromatography).

The effect of the treatment on progression of the tumour is monitored by known techniques.

### EXAMPLE 5

### Intra-Tumoural Acetaldehyde Production As A Novel Anti-Cancer Therapy

The oxidation of ethanol to Acetyl CoA takes place in 2 steps
Step 1: Ethanol+NAD=Acetaldehyde (AA)+NADH

This step is catalysed by the enzyme alcohol dehydrogenase (ADH). This enzyme has many isoforms with differing activity and it is widely distributed in the body at low levels of expression but with very high levels of expression in the liver.
Step 2: Acetaldehyde+NAD=Acetyl-CoA+NADH

This step is catalysed by the enzyme aldehyde dehydrogenase. (ALDH). This enzyme is expressed as a mitochondrial enzyme (ALDH 2) and as a cytoplasmic form ALDH 1. The levels of expression of ALDH 1 vary greatly between tissues with the highest levels being in the liver.

Usually there is a balance between the 2 enzymes and physiological levels of acetaldehyde are low even after ethanol administration.

Ethanol (MW 46) is a well recognised drug with predictable toxicity. The legal blood level for driving is set at 80 mg/dl=17 mM. In contrast to other drugs used as prodrugs, significant ethanol concentrations can be maintained safely with low toxicity.

The purpose of the invention is to produce, for example, intra-tumoural acetaldehyde by delivery of ADH activity to cells that are poorly equipped to metabolise it and so will be liable to chronically accumulate toxic levels of acetaldehyde.

Additionally the toxic effects of acetaldehyde may enhance the cytotoxic activity of conventional chemotherapy drugs.

How Toxic is Acetaldehyde?

Acetaldehyde acts by forming adducts to proteins and by causing DNA strand breaks. There are widely differing reports of the in vitro toxicity of acetaldehyde. All of these reports may significantly underestimate the toxicity of acetaldehyde in vivo as acetaldehyde is very volatile (Boiling Point 22° C.) and is obviously difficult to use in vitro even with sealed culture systems. The ½ life of acetaldehyde in tissue culture at 37° C. is reported to be about 30 minutes (Walia et al (1989) Alcohol Clin Exp Res 13, 766-771).

The toxicity of acetaldehyde in vitro will also vary with the ability of the target cell to metabolise acetaldehyde via the ALDH system and the innate sensitivity of differing cell lines to DNA damaging agents, for example due to differing levels of expression of p53.

The effect of acetaldehyde on cultured cells has been investigated in several papers addressing the effects of alcohol consumption. The effects on cell growth are summarised below.
1. CaCo-2 (Human colorectal cancer cells) (Koivisto & Salaspuro (1998) Carcinogenesis 19, 2031-2036)

500 μM and 1000 μM acetaldehyde for 72 hrs

| | |
|---|---|
| Cytotoxicity | No significant effects. |
| Doubling Time | 500 μm: 150% of control; 1000 μM: 270% of |
| | control |

2. Human Fibroblasts (Grafstrom et al (1994) Carcinogenesis 15, 985-990)
   5 hr exposure to acetaldehyde
   % survival at 1 mM: 75%; at 2 mM: 55%

3. Human Lymphocytes (Bolikle et al (1983) Hum Genet 63, 285-289)

| Concentration | Number of metaphases (cell divisions) counted |
|---|---|
| 0 μM | 200 |
| 90 μM | 200 |
| 180 μM | 200 |
| 360 μM | 200 |
| 720 μM | 130 |
| 1080 μM | 23 |
| 1440 μM | 0 and no growth |

4. Human lymphocytes (Wickramasinghe & Malik (1986) Alcohol Clin Exp Res 10, 350-354)

| | Raji | Molt-4 | WI-L2 | K562 |
|---|---|---|---|---|
| 0 μM | 20 | 29 | 27 | 32 |
| 180 μM | 27 | 35 | 36 | 41 |
| 360 μM | 48 | 46 | 84 | 62 |

The effect of acetaldehyde on Daudi lymphoma cells was investigated. Chronic exposure to acetaldehyde of Daudi lymphoma cells in tissue culture was investigated with the media and acetaldehyde replaced every 24 hours, with the lids either tight closed or slightly open (the norm for tissue culture).

A. Numbers of viable cells×10⁴/ml

| | O hr | 22 hr | 4S hr | 68 hr |
|---|---|---|---|---|
| 0 μM open | 21 | 28 | 40 | 63 |
| 0 μM closed | 21 | 27 | 44 | 66 |
| 100 μM open | 21 | 26 | — | 51 |
| 100 μM closed | 21 | 23 | 28 | 37 |
| 500 μM open | 21 | 22 | 21 | 4 |
| 500 μM closed | 21 | 20 | 18 | 4 |
| 1000 μM open | 21 | 18 | 8 | 2 |
| 1000 μM closed | 21 | 14 | 3 | 0 |

Daudi lyphoma cells may be sensitive to acetaldehyde because they have only limited ALDH activity and so can only metabolise acetaldehyde slowly, and also because lymphoma cells are characteristically sensitive to DNA damaging agents.

The results show that acetaldehyde levels of 1000 μM kill all the cells within 3 days, and at 500 μM nearly all the cells are dead within 3 days. At 100 μM by 68 hours there is no cell death but some inhibition of the rate of growth.

The effect of more prolonged exposure of cells to acetaldehyde was investigated.

B. Repeat method as above changing media and replacing acetaldehyde every 24 hours.

Results are shown as the number of cells×10⁴ remaining viable at each time point. (Days 0-6)

| | acetaldehyde Days | | | | | | |
|---|---|---|---|---|---|---|---|
| | 0/7 | 1/7 | 2/7 | 3/7 | 4/7 | 5/7 | 6/7 |
| 0 μM | 22 | 26 | 42 | 43 | 67 | 71 | 47 |
| 100 μM | 22 | 25 | 28 | 32 | 26 | 19 | 2 (two) |
| 250 μM | 22 | 31 | 25 | 14 | 13 | 0 | — |
| 500 μM | 22 | 31 | 16 | 11 | 3 | 0 | — |
| 750 μM | 22 | 28 | 18 | 5 | 2 | 0 | — |

These results confirm that chronic exposure to acetaldehyde can be cytotoxic with levels as low as 100 μM producing high levels of cell death by 6 days exposure. Higher levels 500 μM and above produce high levels of cell death by 3-4 days exposure.

Combination with Chemotherapy

Following the demonstration that acetaldehyde at achievable therapeutic doses can produce significant cell killing in vitro, the effect of combining acetaldehyde with a conventional cytotoxic agent Cisplatin at 30 μM in vitro was investigated.

The experimental system is similar to that above with the media, cisplatin and acetaldehyde changed every 24 hours.

The results shown are the number of live cells at each time point shown as a % of the number of cells at the start of the expt (ie 100% at time 0 hrs).

| | O hr | 6 hr | 18 hr | 21 hr | 25 hr | 42 hr |
|---|---|---|---|---|---|---|
| Cells alone | 100% | 153% | 229% | 205% | 169% | 270% |
| Cis + 0 | 100% | 147% | 122% | 76% | 82% | 36% |
| Cis + 500 μM | 100% | 129% | 88% | 73% | 55% | 23% |

The experiment was repeated just looking at the +25 hours point, which gives the result below:

| | | | | | | |
|---|---|---|---|---|---|---|
| Cis + 0 | 100% | — | — | — | 63% | — |
| Cis + 500 μM | 100% | — | — | — | 43% | — |

These results show that the addition of acetaldehyde to cisplatin gives an enhanced cytotoxic activity compared to that of cisplatin alone. Of interest there appears to be synergistic activity as the effect of acetaldehyde at 25 hrs (even at 500 μM) on cell numbers when used alone appears to be moderate, with cell numbers in the acetaldehyde monotherapy experiments being similar to or slightly increased compared to 0 hrs.

In Vitro Acetaldehyde Production in Transfected Cells

In the field of alcohol research publications, there are a number of papers that have looked at transfecting cells with ADH and looking at the ability of these cells to make acetaldehyde and the effect of this acetaldehyde on the cells' metabolism.

There is no suggestion in these prior publications that transfection with ADH is useful as therapy.

The reported levels of AA that can be produced by transfecting ADH into cells in vitro and exposing them to ethanol are;
1. Transfected CHO cells: 450 μM (Holownia et al (1999) Brain Res 833, 202-208)
2. Transfected CHO cells: 200-400 μM (Holownia et al (1996) Alcohol 13, 93-97)
3. Transfected CHO cells: 1 mM (after 48 hours) (Mapoles et al (1994) Alcohol Clin Exp Res 18, 632-639)
4. HAD hepatocyte Transfected: 50-140 μM (Clemens et al (1995) Arch Biochem Biophys 321, 311-318)
5. Hela cells via retrovirus: 40 μM (measured in open containers) (Galli et al (1999) Hepatology 29, 1164-1170)
   How Much Acetaldehyde can be Made In Vivo?

Acetaldehyde levels in colonic contents of ethanol fed animals may exceed 3 mM and the cellular cone is considered to reach about 250 μM (Visapaa et al (1998) Alcohol Clin Exp Res 22, 1161-1164).

In the liver during alcohol metabolism, levels in the 100 μM are probably produced (Leieber (1988) Biochem Soc Trans 16, 241-247). Jones et al (1995) Alcohol Alcohol 30, 271-285 reports concentrations of acetaldehyde in the breath after ethanol consumption of 5-1300 nM in different patient groups. The highest levels are in those who took an alcohol dehydrogenase inhibitor (calcium carbimide) with the ethanol. The partition coefficient between blood and air is 190:1 which suggests blood values of 1-260 μM of acetaldehyde. These figures are similar to those reported previously by Jones et al (1987) Alcohol Alcohol s1, 213-217, who reported blood levels of 1.7 to 242 μM in similar experiments.

Liang et al (1999) J Pharmacol Exp Ther 291, 766-772 reports investigation of a transgenic murine cardiac muscle model in which ADH is overexpressed, in order to investigate the mechanism of alcohol cardiomyopathy. In this system ADH activity was increased 40 fold and acetaldehyde levels after ethanol was given IP at 3 g/kg measured at +30 minutes was 70 μM (control heart 15 μM; note cardiac muscle contains high levels of ALDH). Chronic exposure of the mice to ethanol (4%) led to severe myocardial damage at +12 weeks.

Properties of Suitable Forms of Alcohol Dehydrogenase

1. The Km for the ADH Beta2 enzyme, which is suitable for use with the therapeutic methods of the present invention is 0.94 mM. (Km is the concentration at which enzyme activity is 50% of maximal activity). The blood ethanol concentration at driving limit is 17 mM. Therefore it is advantageous that the therapeutic levels of ethanol needed according to the present invention are low and should be sustainable for long periods of time.
2. The Vmax of ADH beta 2 enzyme is 5-40 times greater than that of the other ADH isoenzymes used in the experimental systems for investigating mechanisms of ethanol toxicity described above. (Vmax is the maximal catalytic rate of the enzyme).
   Biochemical Characterisation of Different Forms of ADH

Data from Ehrig et al (1990) AlcoholAlcohol 25, 105-116:

| Class | Km | pH Optimum | Vmax (U/mg) |
|---|---|---|---|
| Human B1 | 49 μM | 10.0 | 9.2 |
| Human B2 | 0.94 mM | 8.5 | 400 |
| Human class IV | 37 mM | | 2,600 |

(Note at pH 7.5 the Vmax of beta 2 is 5-40 times higher that the usual human alpha, beta and gamma variants)

Pyruvate Decarboxylase (PDC)

This enzyme may be used instead of alcohol dehydrogenase to generate acetaldehyde in the vicinity of selected cells. It is present in yeast and some bacteria but not in mammalian cells.

It catalyses the reaction
Pyruvate→Acetaldehyde+C02.

This reaction is used in the production of ethanol by fermentation.

All mammalian cells contain pyruvate as it is a central component of the Krebs cycle.

If the enzyme PDC is introduced into a mammalian cell (for example a cancer cell) in accordance with the present invention it results in the production of significant amounts of acetaldehyde toxic to that cell, as indicated above.

The enzyme is available for yeast such as Saccharomyces. A particularly suitable enzyme is that from Zymomonas, which has suitable stability and high specific activity.

### EXAMPLE 6

### Direct Cytotoxicity and Synergy with Chemotherapy Using Acetaldehyde Production from Ethanol and an Adenovirus Containing Human Beta 2 Alcohol Dehydrogenase

Introduction

The use of antibody (ADEPT) and gene (GDEPT) directed enzyme-prodrug systems to selectively produce cytotoxic agents within malignant or other target cells is well established in experimental therapeutics. A number of pro-drug toxin systems including; nitroreductase, cytosine deaminase, horse radish peroxidase, thymidine kinase and cytochrome p450 have been described that all generate toxic agents on exposure to their pro-drugs.

To investigate an alternative approach to the GDEPT system based on the prolonged exposure to a cytotoxic agent which could be used in combination with conventional cytotoxic agents we have examined the use of acetaldehyde generated from the metabolism of ethanol mediated by alcohol dehydrogenase (ADH).

Acetaldehyde is a well recognized toxin producing single DNA breaks (Singh and Khan 1995) and forming protein adducts (Kolber and Terabayashi 1991). Whilst not previously described as a potential therapeutic agent, the ability of prolonged acetaldehyde exposure to damage cell, inhibit their growth and be directly cytotoxic in vitro and in vivo is well characterized (Clemens 2002)

The metabolism of ethanol takes place in two major steps. Initially ethanol is converted to acetaldehyde via the enzyme alcohol dehydrogenase, predominantly within hepatocytes. The acetaldehyde produced is rapidly converted to Acetyl-Coa via the action of aldehyde dehydrogenases (ALDH) predominantly in mitochondria but also by the cytoplasmic ALDH in hepatocytes (Klyosov et al 1996, Salispuro Oxford Textbook of Medicine).

As a result of the high level of expression of both enzymes systems within ethanol metabolizing cells, the accumulation of acetaldehyde is restricted and cell damage usually only occurs on protracted exposure. However the potential to use gene therapy delivery and expression systems to selectively enhance ADH activity in cells with naturally low levels of ALDH may allow the production of elevated levels of acetaldehyde. Exposure to for prolonged periods to these enhanced acetaldehyde concentrations may advantageously lead directly to cell death or promote additional cytotoxicity in targeted cells when exposed to conventional chemotherapy agents.

To demonstrate intra-cellular over production of acetaldehyde as a therapeutic approach we have constructed an adenovirus containing the human beta 2 ADH gene. This allele is a common variant of the more frequent human B1 ADH often referred to as the oriental allele. The enzyme characteristics, including a pH optimum of 8.5, a Vmax approximately 40-80 fold higher than the B1 enzyme and a Km for ethanol of 0.94 mM (Yoshida 1981, Bosron 1985) make it an effective option to produce acetaldehyde at a high rate using ethanol at physiologically sustainable concentrations.

The pharmacokinetics and metabolism of ethanol have been extensively investigated and the toxicities short term and chronic well described. Ethanol is freely soluble in water and distributes rapidly to the interior of cells by diffusion. The ability of humans to tolerate levels of ethanol in excess of the Km of the Beta 2 ADH enzyme for protracted periods of time is documented in a number of publications and numerous anecdotal reports. To give perspective on the concentrations of ethanol described, the current UK driving limit of 80 mg/dl equates to approximately 17 mM.

Materials and Methods for Example 6

Cell Lines and In Vitro Acetaldehyde Toxicity Assays

Standard tissue culture techniques were used to maintain Hela, Daudi, Jurkat, CMT-64 cells in flasks in DMEM+10% FCS in a 37° C. incubator.

Cell lines subcloned into 25-cm² flasks and incubated in media containing dilutions of acetaldehyde, cisplatin or both. Flasks remained sealed during incubation and the media and acetaldehyde/cisplatin were replaced daily. Viable cell counts were made using Trypan blue exclusion and a standard haemocytometer.

ADH Containing Adenovirus (Ad-ADH) Construction

A human ADH beta 2 cDNA clone previously constructed for prokaryotic protein expression (kind gift of Dr T Hurley, Indiana University) was used to construct the Ad-ADH. After the re-introduction of the Kozak consensus sequence by PCR primer addition the integrity of the construct was confirmed by sequencing, cloned into pShuttle-CMV as a HindIII-XbaI fragment and then transferred into replication deficient adenovirus serotype 5 using homologous recombination in E.Coli. Ad-ADH production was completed in human embryonic kidney 293 cells following the method described by He (He et al 1998) and purified by CsCl banding.

In Vitro Ad-ADH Ethanol Mediated Toxicity

Cells were placed into 6 well plates at 5×10⁶ per well. After overnight incubation, the cells were washed twice in PBS then incubated with virus (Hela MOI 100, CMT-64 MOI 1000) at 37° C. for 1 hr in 1 ml of serum free media. Following addition of 3 mls more of tissue culture medium the cells were incubated overnight, assayed for ADH activity and used for experiments as below.

ADH Activity Spectrophotometer Assay

The ADH activity of native and adenovirus transfected cells was assessed using a spectrophotometer assay following the method outlined by Crow et al (ref). In brief, approximately 10⁶ cells were lysed in 1 ml of 0.05M Pyrophosphate buffer pH 8.8 containing 0.1% Triton X-100. 400 uL of lysate was added to 800 ul of ADH assay buffer comprising 0.025M pyrophosphate buffer, 7.5 mM NAD and 5% Ethanol The change in the absorbance at 340 nM was recorded on a spectrophotometer.

Ad-ADH Ethanol Cytotoxicity Assays

5×10⁵ cells were seeded into 25-cm² flasks and grown in 5 ml of tissue culture media and dilutions of ethanol, the ADH inhibitor 4-methylpyrazole (4-MP) 10 uMol (Sigma, Poole, UK) cytotoxic drugs. The lids to these flasks were screwed shut and the numbers of cells in replicates of the experiment were counted at intervals by Trypan blue exclusion and a haemocytometer.

1/ In Vitro Acetaldehyde and Cisplatin Mediated Toxicity

The effects of prolonged acetaldehyde exposure on a number of cell lines are demonstrated in FIG. 1. (In 1a Daudi, 1b Jurkat, 1c Hela and 1d CMT-64.) A reduction in the rate of proliferation is demonstrated for all of the cell lines at 250 uM acetaldehyde, the lowest concentration examined. The effect of increasing concentrations is is demonstrated with both increasing growth inlibition and evidence of cell killing at the higher concentrations at 500 uM and 1 mM. This effect is most marked in the lymphoid cell lines Daudi and Jurkat where the viable cell numbers fell to only 37% and 7% of their starting number respectively by day 3 and with no Jurkat cells viable by day 4.

In FIG. 2 the effects of exposure to both acetaldehyde and cisplatin are demonstrated. Using Daudi cells at a higher cell density the effects of acetaldehyde alone, are less than in FIG. 1 but show a reduction in cell number to 67% of the starting number after 3 days exposure, whilst cisplatin reduces their number to 21%. The two agents in combination reduce the cell count to only 4.8% of their starting number. Similarly the results for the CMT-64 cells also show an additive effect from exposure to both drugs with acetaldehyde alone restricting growth to 170% of the starting number, cisplatin reducing to 80% and the combination reducing further to 65%.

2/(Ad-ADH) Construction

The integrity of the amended ADH beta2 cDNA construct prior to incorporation in the adenovirus was confirmed by DNA sequencing. The sequences was confirmed as correct by comparison with those obtained by others in the field and this amended sequence may be found in the GenBank sequence database.

The correct size and function of the ADH protein produced by the adenovirus is confirmed by western blotting of infected cells and comparison of functional ADH activity of native and Ad-ADH infected CMT-64 cells. The enzymatic conversion of ethanol to acetaldehyde as measured by NADH production in a spectrophotometer assay demonstrated that the native CMT-64 cells showed little activity. The OD 340 nm with untreated cells increased from 0.00 at 1 minute to 0.056 at 30 minutes, whilst the Ad-ADH CMT-64 cells showed an increase from 0.0 to 0.77 during the same period.

Ad-ADH Ethanol Cytotoxicity Assays

The effect of ethanol exposure on wild type and Ad-ADH infected cells CMT-64 cells is demonstrated in FIG. 3. With the wild type CMT-64 cells the addition of ethanol (20 mM), 4-MP or both has no significant effect on cell numbers with increases in cell numbers from 5×10⁵ on day 0 to 12-13.5×10⁵ on Day 2. In contrast with the Ad-ADH CMT-64 cells the addition of ethanol reduces the number of cells to 3.7×10⁵ compared to 12.4×10⁵ for those not exposed to ethanol. In addition to reducing proliferation the reduction in the total cell number to only 74%, of the starting number, on day 2 and a further reduction to 36% by day 4 demonstrates that cell death as well as growth inhibition has occurred.

In FIG. 4 show the results of extended ethanol exposure to Ad-ADH CMT-64. By day 4 the Ad-ADH cells not exposed to ethanol have increased 14.6×10⁵ whilst those exposed to ethanol have reduced in number to 1.8×10⁵ only 36% of their starting number.

Combined Effects of Ethanol and Cisplatin on Ad-ADH Cells

The results of combining ethanol/acetaldehyde-mediated cytotoxicity with the cytotoxic drug Cisplatin are shown in FIG. 4. Ad-ADH CMT-64 cells seeded at 5×10⁵ on Day 0 reached 12×10⁵ by day 2, whilst the numbers of cells in flasks exposed to ethanol, cisplatin and both fell to 3.5×10⁵, 3.75×10⁵ and 2.2×10⁵ respectively. Similarly Ad-ADH Hela cells seeded at 5×10⁵ on Day 0 reached 14.5×10⁵ by day 2, whilst the numbers of cells in flasks exposed to ethanol, cisplatin were 6.2×10⁵, 3.2×10⁵ and 1.8×10⁵respectively

In Vivo Growth Inhibition/In Vivo Ad-ADH Ethanol Mediated Toxicity

CMT-64 cells are injected s-c into mice and tumour growth allowed to proceed.

Virus described above is introduced into the CMT-64 cells.

Alcohol (ethanol) is injected IP for several days.

Tumour dimensions are monitored thereafter.

Following the above approach, significant inhibition of growth in the experimental group compared to the controls particularly on Day 6 can be observed in FIG. 5.

In Vivo Growth Inhibition/In Vivo Ad-ADH Ethanol Mediated Toxicity—Direct Injection

CMT-64 cells are injected s-c into mice and tumour growth allowed to proceed.

Virus described above is injected per tumour.

Alcohol (ethanol) is injected IP for several days.

Tumour dimensions are monitored thereafter.

Repeat injections of virus may be given as necessary.

In Vivo Growth Inhibition/In Vivo Ad-ADH Ethanol Mediated Toxicity

The invention is now demonstrated in more detail.

Method

In Vivo Assessment of Ad-ADH Ethanol Mediated Toxicity

The animal experiment was approved by the Cancer Research UK animal use and safety committee and the Home Office before initiation. On the day prior to injection, 6×10⁶ CMT-64 cells were transduced with the Ad-ADH virus in vitro, then on Day 0 adult C57B/6 mice received s-c injections in the flank of 5×10⁵ Ad-ADH CMT-64 cells (groups 1 and 2) or mock infected CMT-64 cells (groups 3 and 4) suspended in 100 uL of PBS. On days 0-4 groups 1 and 3 were injected with 500 ul of 12% ethanol (2 g/kg) solution IP and groups 2 and 4 received 500 uL of water IP, there were 6 mice in each group. Tumour volume was estimated twice weekly using the formula: volume=(length×width×width)×(3.142/6).

Results: In vivo growth inhibition

The results of this in vivo experiment are shown in FIG. 6. The administration of ethanol or water appears to have had no measurable effect on the growth of the wild type CMT-64 cells. The mean tumour volumes on days 6 and 13 were 0.1645 cm³ and 0.514 cm³ for the ethanol treated mice (group 3) compared to 0.1455 cm³ and 0.536 cm³ for the water treated controls (group 4).

In contrast with the CMT-64 cells transduced with Ad-ADH the mean size of the tumours in the mice exposed to ethanol was significantly smaller at 0.036 cm³ (range 0.008-0.100 cm³) on day 6 and 0.18 cm3 (0.058-0.292 cm³) on day 13 compared to 0.121 cm³ (0.048-0.199 cm³) and 0.431 cm³ (0.254-0.638 cm³) on those days for the control mice bearing Ad-ADH CMT-64 cells exposed to water (p=>0.001).

The experiment was halted on day 14, when the mice with the larger tumours were sacrificed. Within the Ad-ADH ethanol group one mouse was continued through to Day 23 with no further increase in tumour size which measured 0.058 cm3 on Day 10 and Day 23.

FIG. 6 shows the in vivo effects of 5 days I-P ethanol or water administration on the growth of Ad-ADH and native CMT-64 cells implanted s-c in C57 mice. Tumour size was measure on Days 6, 10 and 13; the results are shown as the mean and SD from 6 mice per group.

Thus it can be seen that treatment according to the present invention is effective in damaging target cells, preferably killing them, in a subject.

### Discussion

Using ethanol and a high activity variant of human alcohol dehydrogenase we have demonstrated a different approach to these systems with a pro-drug toxin combination that is capable of the prolonged in vivo production of the toxic agent.

Acetaldehyde, the main metabolite of ethanol, is a well recognized toxin and carcinogen with previous in-vitro studies demonstrating that significant effects on cell proliferation and induction of apoptosis occur on chronic exposure (Wickramasinghe Clemens 2003). The results in this current study shown in FIG. 1 confirm these findings with growth inhibition occurring in all 4 cells lines tested at levels of 250 uM and significant cytotoxicity particularly in the lymphoid Daudi and Jurkat cells lines with chronic exposure to acetaldehyde at concentrations above 500 uM.

Our experiments confirm sensitisation of cancer cells to the effects of conventional cytotoxic agents as shown in FIG. 2, where the exposure of Daudi cells and CMT-64 cells to the combination of cisplatin and acetaldehyde in accordance with the present invention resulted in significantly greater cell killing than to either drug alone.

To examine the potential of acetaldehyde production to be used for therapeutic purposes we have constructed an adenovirus (Ad-ADH) containing the beta 2 ADH gene, which has a maximum enzymatic rate (Vmax) approximately 40-80 fold high than the usual human ADH enzymes (Bosron et al), and at least 16 fold higher than the usual human ADH enzymes at ‘physiological’ ethanol concentrations.

The introduction of this virus into cells, which have naturally low levels of the acetaldehyde metabolizing enzyme ALDH, and their exposure to ethanol produces intra-cellular and local concentrations of acetaldehyde that are directly cytotoxic and/or sufficient to enhance the cytotoxic activity of conventional cytotoxic agents.

The effects of the enhanced ethanol metabolism and acetaldehyde production on the growth and viability delivered by the Ad-ADH, were examined in the comparison of the effects of 2 days ethanol exposure on wild type and Ad-ADH CMT-64 cells seen in FIG. 3. Whilst the growth of the wild type CMT-64 cells is unaffected by ethanol exposure, the growth of the Ad-ADH cells is significantly affected, falling to 74% of their starting number and numbering only 30% of their non-ethanol exposed controls. Similar results were demonstrated in a 4 day prolonged exposure of Ad-ADH infected CMT-64 cells to ethanol which resulted in a reduction in the total cell number to only 36% of their starting number. The blocking of this effect by the ADH inhibitor 4-MP confirms the role of acetaldehyde production in producing this effect.

The ability of cisplatin exposure to add to the cytotoxic effects of endogenously generated acetaldehyde is demonstrated in FIG. 4. Here both Ad-ADH CMT-64 and Ad-ADH Hela cells show reduced viability after 2-3 days exposure to ethanol and cisplatin that compared to either drug alone.

Thus the present invention can be used to produce major effects and significant acetaldehyde mediated cell death in vitro.

However the accurate assessment of the cytotoxic effects of prolonged exposure to acetaldehyde is extremely difficult to quantify in vitro. Acetaldehyde is volatile (bp 21° C.) rapidly evaporating with a T1/ of 30 minutes from tissue culture conditions (Walia et al 1989). Additionally all cells have some ability to metabolise acetaldehyde so resulting in more rapid reductions in acetaldehyde concentrations dependant on the type and the number of cells present.

This effect of cellular acetaldehyde metabolism is the likely explanation for the difference in cytotoxic effects seen when varying numbers of cells were used in the experiments shown in FIG. 1 and FIG. 2.

Head space gas chromatography can be used to verify acetaldehye concentrations.

To obtain more clinically meaningful data we examined a simple pre-clinical model, that examined the growth and viability of Ad-ADH CMT-64 cells in mice exposed to ethanol for a 5 day period.

The results shown in FIG. 6 demonstrate that administration of ethanol to C57 mice bearing wild type CMT-64 cells has no significant effect on their tumour growth. In contrast the administration of ethanol to the mice with Ad-ADH CMT-64 cells produced a significant delay in tumour growth with the median size of the ethanol treated mice being 30% of the size of the water treated controls on day 6 and 42% on day 13. Of interest the tumour size in one of the experimental group remained stable at 0.058 cm³ through to day 23. It is likely that this system can be improved further by optimising ethanol levels. For example, it is probable that mice received less than optimal ethanol exposure for continual ADH activity since available suggests that mice metabolise ethanol 5 times faster than humans. Studies in C57 mice have demonstrated that blood ethanol levels fell to below the limit of detection within 6-7 hours after IP ethanol injections of 3.7-3.8 g/kg. This would suggest that the mice in this example, which only received 2 g/Kg, had therapeutically useful blood ethanol concentrations for only a few hours each day (approx 5 hours per day), so receiving 5 short pulses of acetaldehyde rather than more optimal continual exposure. Thus, greater and/or more frequent administrations will be beneficial depending upon the subject size and rate of metabolisation/elimination. Such manipulation of the ethanol levels is within the abilities of a person skilled in the art in conjunction with the teachings presented herein.

Whilst measuring the concentration of acetaldehyde produced within the Ad-ADH transduced cells in vivo was not performed, it is probable that levels in the 500 uM to 1 mM range were produced. This would be supported by the in vitro data that showed significant growth inhibition of CMT-64 cells occurred at concentrations of over 500 uM. Additionally clinical data from volunteers consuming alcohol in conjunction with an hepatic ALDH inhibitor, showed systemic concentrations of acetaldehyde of up to 240 uM. With the considerable extrahepatic dilution this would suggest that intrahepatic and intrahepatocyte concentrations are considerably higher, and therefore would be correspondingly high inside the target cells of the present invention such as those shown herein.

In addition to the direct toxic effects, acetaldehyde exposure can also produce immunological effects that may be therapeutically useful. The action of acetaldehyde on proteins produces alterations in their structure that allows acetaldehyde damaged cells to be recognised by both autoantibodies and cytotoxic T cells. These mechanisms, which are similar to those involved in the pathogeneses of alcoholic cirrhosis, may offer a chance for up regulation by vaccination so producing effective immune responses that distinguish acetaldehyde damaged tumour cells from healthy non-malignant cells.

Preferably a high proportion of target tumour cells are transduced.

The relative lack of clinically significant toxicity between alcohol and the majority of chemotherapy agents suggests that this approach to enzyme prodrug therapy could safely be combined with most conventional chemotherapy drugs, advantageously with synergistic results as shown in FIG. 4.

Systemic acetaldehyde toxicity is unlikely to occur, whilst intra-tumoural concentrations of over 1 mM may be generated, the effect of venous dilution and rapid hepatic metabolism would reduce concentrations below the 40-60 uM level described for systemic acetaldehyde toxicity.

This system allows prolonged action, is effective against both dividing and non-dividing cells and is safe and simple to administer as a monotherapy or in conjunction with conventional chemotherapy drugs.

Furthermore, a replicating adenovirus and/or higher and/or more prolonged ethanol exposure should enhance the effects.

The production of cell damaging cytotoxic levels of acetaldehyde in targeted cells is a new approach to enzyme pro-drug based therapies.

With a high expression adenovirus system, the use of ethanol and the high activity beta 2 variant of ADH the system allows the production of therapeutic differential between the very low levels of hepatic toxicity from acetaldehyde production in the livers and the high levels produced locally at cells poorly equipped to metabolise it. Whilst systemic acetaldehyde toxicity can occur, the use of this system would be very unlikely to reach these systemic levels due to the dilution effects, plasma protein binding and near 100% removal on passage through the liver.

The lack of clinically significant toxicity between alcohol and the majority of chemotherapy agents (Brown et al 2003) further confirms that this approach to enzyme prodrug therapy can safely be combined with conventional chemotherapy drugs.

The ability to selectively express this enzyme and so produce acetaldehyde within malignant, virus infected or other diseased cells may allow selective cytotoxicity to be effected with an economic, simple and tolerated system either as a monotherapy or in combination with conventional cytotoxic agents.

### EXAMPLE 7

### Target Cell Cytotoxicity from Intracellular Acetaldehyde Production Using Pyruvate Decarboxylase

A synthetic cDNA of the gene pyruvate decarboxylase (PDC) from the bacterium Zymomonas Palmae is produced. This work including addition of a Kozak consensus sequence is performed by oligo nucleotide synthesis by Entelechon Ltd.

This forms a cDNA suitable for expression in a eukaryotic/mammalian system. An adenovirus for eukaryotic expression of the PDC cDNA is constructed. This uses the inducible expression vector system: pAdenoVator-CMV(CuO) which is provided by Qbiogene Ltd.

The cytotoxic effects of PDC expression in Eukaryotic cells are demonstrated.

Infecting cells:

Using the adenovirus PDC construct (Ad-PDC) at a MOI of 10-1000:1, the cancer cell line CMT-64 is infected using this method:

Plate CMT-64 cells into 6 well plates at 5×10⁵/well in 4 mls of media

Allow cells to become adherent

Wash media off cells

Add virus suspension in minimal volume of media without FCS.

Incubate for 1 hr at 37° C.

Add normal media to 4 mls

Protein expression is induced and enzymatic activity is measured. Using the Qbiogene system the transcription of the gene is commenced by the addition of ‘Cumate’.

Measurement of protein activity is assayed by spectrophotometer using the method of Neale et al:
Add cell lysate sample to 2 ml of 5 mM MgCl2 in 50 nM Mes-KOH buffer and thiamin diphosphate at 100 uM.
Add 60 uL of 172 mM Sodium pyruvate, 5.15 mM NADH and 340 IU/ml alcohol dehydrogenase.
Measure OD at A340 at 25° C.

An increasing OD240 over a period of approx. 30 minutes in the infected samples indicates increasing enzyme activity.

Measurement of Acetaldehyde Production/Concentration

Using headspace gas chromatography the accumulation of acetaldehyde in tissue culture media from the action of Ad-PDC on endogenous pyruvate is measured.

Over 24 hours a steady escalation in acetaldehyde is advantageously achieved, preferably approaching 0.8 uM.

Measurement of Effects on Proliferation In Vitro

Method
1/Add 1×10⁶ native or Ad-PDC infected cells to small tissue culture flask
2/Add Cumate inducer
3/Grow cells for 3 days and then count by trypan blue exclusion.

Expansion of the native cells towards 6×10⁶ over four days compared with a parallel decline in infected cells towards background levels indicates a robust killing effect.

Effect on Growth In Vivo

Method

Inject either native or Ad-PDC CMT-64 cells (1×10⁶) s-c into C57 mice.

Measure tumor diameter daily.

An increase in native tumour diameter from approx. 5 mm towards approx. 20 mm at day 9 compared with a decrease in tumour diameter from 5 mm towards 1 mm or even less at day 9 indicates an excellent in vivo killing effect.

1. A method of damaging target cells in a subject, the method comprising administering to the subject
(1) a nucleic acid encoding a compound capable of converting a substrate to acetaldehyde, wherein said compound is an enzymatically active portion of alcohol dehydrogenase; and
(2) a substrate which is converted to acetaldehyde by the portion capable of converting said substrate to acetaldehyde;
wherein said substrate is ethanol.

2. A method according to claim 1 further comprising administering a component that is capable of inhibiting aldehyde dehydrogenase.

3. A method according to claim 2 wherein said component that is capable of inhibiting aldehyde dehydrogenase is Disulfiram.

4. A method according to any one of claims 1 to 3 wherein the nucleic acid is in the form of a viral vector.

5. A method according to claim 4 wherein the viral vector is a DNA based viral vector.

6. A method according to claim 5 wherein the DNA based viral vector is an adenovirus derived viral vector.

7. A method according to any one of claims 1 to 3 wherein the nucleic acid comprises a polynucleotide comprising a target cell-specific promoter operably linked to a polynucleotide encoding said alcohol dehydrogenase.

8. A method according to claim 1 in which the portion of alcohol dehydrogenase converts the ethanol to acetaldehyde as a result of its enzymatic activity.

9. A method according to claim 4 wherein said vector comprises a target cell specific portion.

10. A method according to claim 9 in which the target cell specific portion comprises an antibody or part thereof.

11. A method according to claim 9 in which the target-cell specific portion is capable of selectively binding to a cell surface entity.

12. A method according to claim 11 in which the cell surface entity is a tumour-associated antigen.

13. A method according to claim 9 in which the target cell specific portion comprises a liposome.

14. A method according to claim 1 further comprising administering radiation therapy is also administered to the subject.

15. A composition comprising a compound as defined in claim 1, wherein the portion of alcohol dehydrogenase in an enzymatically active portion of human alcohol dehydrogenase.

16. A composition according to claim 15 wherein said human alcohol dehydrogenase is alcohol dehydrogenase β2.

17. A composition according to claim 15 or claim 16 further comprising a substance which is capable of inhibiting aldehyde dehydrogenase.

18. A composition according to claim 17 wherein said substance which is capable of inhibiting aldehyde dehydrogenase is Disulfiram.

19. A composition according to any one of claims 15 or 16 further comprising a chemotherapeutic agent.

20. A composition according to any one of claims 15 or 16 further comprising an immunosuppressive agent.

21. (canceled)

22. A method for the treatment of cancer comprising administering to a subject in need thereof a composition of any one of claims 15 or 16 in an amount effective to treat said cancer.

23. A method of treating cancer comprising administering ethanol or pyruvate to a subject in need thereof.

24. A therapeutic system or kit comprising a compound or system as defined in claim 1, or a composition as defined in claim 15 or 16, and a second component which comprises ethanol, and optionally a third component that is capable of inhibiting aldehyde dehydrogenase.

25. A therapeutic system or kit according to claim 24 in which the aldehyde producing portion is a catalytically active portion of alcohol dehydrogenase, the second component is ethanol and the third component is Disulfiram.

26. (canceled)

27. (canceled)

28. A composition according to claim 19 further comprising an immunosuppressive agent.

29. A composition according to claim 17 further comprising a chemotherapeutic agent.

30. A composition according to claim 18 further comprising a chemotherapeutic agent.

31. A composition according to claim 17 further comprising an immunosuppressive agent.

32. A composition according to claim 18 further comprising an immunosuppressive agent.

33. A composition according to claim 19 further comprising an immunosuppressive agent.

34. A method for the treatment of cancer comprising administering to a subject in need thereof a composition of claim 17 in an amount effective to treat said cancer.

35. A method for the treatment of cancer comprising administering to a subject in need thereof a composition of claim 18 in an amount effective to treat said cancer.

36. A method for the treatment of cancer comprising administering to a subject in need thereof a composition of claim 19 in an amount effective to treat said cancer.

37. A method for the treatment of cancer comprising administering to a subject in need thereof a composition of claim 20 in an amount effective to treat said cancer.
